(19) United States
(12) Patent Application Publication
(10) Pub. No.: US 2014/0148353 A1
(43) Pub. Date: May 29, 2014

(54) PROTEIN EXPRESSION-BASED CLASSIFIER FOR PREDICTION OF RECURRENCE IN ADENOCARCINOMA

(71) Applicants: David L. Rimm, Branford, CT (US); Valsamo Anagnostou, New Haven, CT (US)

(72) Inventors: David L. Rimm, Branford, CT (US); Valsamo Anagnostou, New Haven, CT (US)

(73) Assignee: YALE UNIVERSITY, New Haven, CT (US)

(21) Appl. No.: 14/000,821

(22) PCT Filed: Feb. 22, 2012
(86) PCT No.: PCT/US12/26201

§ 371 Date: Jan. 3, 2014

Related U.S. Application Data
(60) Provisional application No. 61/463,715, filed on Feb. 22, 2011.

(51) Int. Cl. G01N 33/574

(52) U.S. Cl. CPC G01N 33/57449
USPC 506/9; 435/7.1

(57) ABSTRACT

A method for making a prognosis for a patient afflicted with a type of cancer which comprises (a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; (b) calculating a score based on the levels of expression determined in step (a); (c) correlating the score obtained in step (b) with a series of predetermined reference scores associated with a series of prognoses; so as to thereby make a prognosis for the patient.

This application claims priority of U.S. Provisional Application No. 61/463,715, filed Feb. 22, 2011, the entire content of which is hereby incorporated by reference into this application.

This invention was made with support under Grant No. 2009-0097 from the Connecticut Department of Health.

Throughout this application, various publications are referenced by footnotes and/or parentheses. The disclosures of each of the publications found in this specification is hereby incorporated by reference into this application in order to more fully describe the state of the art as known to those skilled therein as of the date of this application.

### FIELD OF THE INVENTION

This invention relates to the field of predicting recurrences of adenocarcinoma and stratifying low stage lung adenocarcinoma patients into groups that will or will not benefit from adjuvant therapy.

### BACKGROUND OF THE INVENTION

Lung cancer, predominantly nonsmall-cell lung cancer (NSCLC), is the most common cause of death from cancer worldwide, with 226,160 new cases and 160,340 deaths due to disease estimated to occur in 2012 in the US alone (National Cancer Institute). Survival rates, depending on stage at diagnosis range from 49% to 16% to 2% for local regional and distant stage disease respectively (Ries L, Eisner M, Kosary C, et al., eds.: Cancer Statistics Review, 1975-2002. Bethesda, Md.: National Cancer Institute, 2005). Screening patients for early detection of disease has not been shown to impact mortality (Bach P B, Silvestri G A, Hanger M, et al.: Screening for lung cancer: ACCP evidence-based clinical practice guidelines (2nd edition). Chest 132 (3 Suppl): 69S-77S, 2007), perhaps due in part to current treatment paradigms. Early stage treatment includes surgical resection but no adjuvant chemotherapy, resulting in poor overall 5 year survival rate, generally due to recurrence of disease (Mountain CF. The international system for staging lung cancer. Semin Surg Oncol 2000; 18; 106-115). If those patients at greatest risk for recurrence could be identified, more aggressive treatment could be pursued. For example adjuvant chemotherapy could be considered for those early stage patients that could be subclassified as having a particularly poor prognosis.

Immunohistochemistry (IHC) is often used to assess the expression and localization of biomarkers in tumor specimens; however this technique has several shortcomings. IHC is generally poorly standardized and results are typically evaluated by eye in a subjective manner. However, quantitative IHC (qIHC), often based on image analysis, for example AQUA technology can be used to obtain highly standardized, reproducible and quantitative measurements of biomarkers in situ. (U.S. Pat. No. 7,219,016; and publications US2009/0034823 and US2010/136549).

Biomarkers having prognostic potential for lung cancer were recently reviewed (Zhu, et al Immunohistochemical markers of prognosis in non-small cell lung cancer: a review and proposal for a multiphase approach to marker evaluation. J Clin Pathol 2006; 59:790-800). In this review biomarkers EGFR, HER2, Ki67, p53 and Bcl-2 were reported to have shown prognostic potential in the literature but have not been proven to have clinical value. Furthermore, p27^{kip1}, VEGF A, Cyclin E and p16^{INK4A} were considered promising but requiring further study.

STAT3: Signal transducer and activator of transcription 3 (STAT3) is phosphorylated by receptor associated kinases in response to cytokines and growth factors, and then act as a transcription activator, impacting cell growth and apoptosis. STAT3 is known to promote oncogenesis (Klampfer L., 2006 “Signal transducers and activators of transcription (STATs): Novel targets of chemopreventive and chemotherapeutic drugs”. Curr Cancer Drug Targets 6 (2): 107-121; Alvarez J V et al 2006, “Signal transducer and activator of transcription 3 is required for the oncogenic effects of non-small-cell lung cancer-associated mutations of the epidermal growth factor receptor”, Cancer Res 66 (6): 3162-3168), but also has been reported to function in a tumor suppressor role. (de la Iglesia N, et al., 2008 “Identification of a PTEN-regulated STAT3 brain tumor suppressor pathway”, Genes Dev. 22 (4): 449-462).

Cyclin D1: Cyclin family members are critical players in cell cycle progression, and thereby frequently associated with cancer and tumorigenesis. Cyclin D1 is associated with G1 progression and known to be upregulated in lung cancer (Kim J K et al, Nuclear cyclin D1: an oncogenic driver in human cancer. J Cell Physiol 2009; 220:292-296.) but was deemed not statistically significantly associated with poor prognosis (Zhang et al, 2011 Clinical Lung Cancer doi:10.1016/j.cllc.2011.20.003).

TTF1: Thyroid transcription factor 1 is commonly used as a marker for lung tumors that when positive generally indicates a tumor is of the adenocarcinoma type. TTF1 has also been found to be an independent marker of good prognosis in adenocarcinoma lung cancer patients (Perner S, et al 2009 J Pathol 217:65-72).

Beta catenin: Beta catenin, a member of the Wnt signaling pathway regulates epithelial cell growth and adhesion. Loss of beta catenin is associated with poor prognosis in lung cancer patients. (Kase S et al, Clin Cancer Res 2000; 6:4789-4796)

A critical clinical problem in management of adenocarcinoma of the lung is to determine which low stage patients are cured by surgery alone, versus which will benefit from adjuvant chemotherapy. While there are only 20% of all lung cancer patients diagnosed at low stage, that still represents over 30,000 patients in the United States.

This invention is a protein-based test that can stratify low stage lung adenocarcinoma patients into groups that do or do not benefit from additional therapeutic treatment such as chemotherapy.

The new process assesses the level of four (a) key proteins using, for example, the AQUA technology method of standardized quantitative immunofluorescence IHC as previously described (Camp et al 2002 Nature Medicine 8(11)1323-1327, U.S. Pat. No. 7,219,016; Gustavson et al AQUA Technology and Molecular Pathology in Pathology in Drug Discovery and Development, Platero ed. John Wiley & Sons, Inc, Hoboken, N.J. 2009). The method may be used on formalin-fixed, paraffin-embedded tumor specimens, fine-needle aspirates, or other histological samples.

The four proteins that are measured are:

Thyroid Transcription Factor-1 (TTF1), Signal transducer and activator of transcription-3 (STAT3), Beta-Catenin, and Cyclin D1.

These are measured in a quantitative manner using standard curves to assure accuracy and precision in measurement. From these data a risk score is then calculated.

Patients can then be divided into either high risk or low risk groups based on cut-points determined from previous studies. Stage 1 lung cancer patients with adenocarcinoma in the low risk group have a 95% chance of survival at 5 years, compared to a 40% chance in the high risk group. Although stage I patients are not usually given chemotherapy or other therapeutic treatment, utilizing this assay and algorithm a study would select a subset (as many as 66% of patients) that fall into the high risk group that would then benefit from additional therapeutic treatment such as chemotherapy.

### SUMMARY OF THE INVENTION

The invention provides a method for making a prognosis for a patient afflicted with a type of cancer which comprises: a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; b) calculating a score based on the levels of expression determined in step (a); c) correlating the score obtained in step (b) with a series of predetermined reference scores associated with a series of prognoses; so as to thereby make a prognosis for the patient.

The invention provides a method for classifying a patient diagnosed with cancer as being at a low risk for a recurrence of cancer comprising: a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; b) calculating a score based on the levels of expression determined in step (a); and c) comparing the score obtained in step (b) with a predetermined reference score cutpoint separating high risk from low risk patients; wherein the patient is at a low risk of developing a recurrence of cancer if the score obtained in step (b) is less than the predetermined reference score cutpoint.

The invention provides a method for classifying a patient diagnosed with cancer as being at a high risk for a recurrence of cancer comprising: a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; b) calculating a score based on the levels of expression determined in step (a); and c) comparing the score obtained in step (b) with a predetermined reference score cutpoint separating high risk from low risk patients; wherein the patient is at a high risk of developing a recurrence of cancer if the score obtained in step (b) is greater than the predetermined reference score cutpoint.

A method for determining the likelihood that a patient diagnosed with cancer will develop a recurrence of cancer comprising: a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with high risk and low risk patients; so as to thereby determine the likelihood that the patient will develop a recurrence of cancer.

A method for determining the likelihood that a patient diagnosed with cancer will benefit from adjuvant therapy comprising: a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with a series of high risk and low risk patients; so as to thereby determine the likelihood that the patient will benefit from adjuvant therapy.

A method for determining the likelihood that a patient diagnosed with cancer will benefit from adjuvant therapy comprising: a) determining in a sample of a tumor from the patient a level of expression for thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with a series of high risk and low risk patients; so as to thereby determine the likelihood that the patient will benefit from adjuvant therapy.

A method for determining the likelihood that a patient diagnosed with cancer will benefit from adjuvant therapy comprising: a) determining a total level of expression of cyclin D1 within a nuclear compartment and non-nuclear compartments combined in cells of interest in a tumor sample from the patient; b) determining a total level of expression of signal transducer and activator of transcription-3 (STAT-3) within the nuclear compartment and the non-nuclear compartments combined in cells of interest in the sample from the patient; c) determining a level of expression of thyroid transcription factor-1 (TTF1) in the nuclear compartment in cells of interest in the sample from the patient; d) determining a level of expression of beta-catenin in a cytoplasmic compartment in cells of interest in the sample from the patient; e) calculating a score based on the levels of expression determined in steps (a) through (d); and f) correlating the score obtained in step (b) with a series of predetermined reference scores associated with a series of high risk and low risk patients; so as to thereby determine the likelihood that the patient will benefit from adjuvant therapy.

A method for determining the likelihood that a patient diagnosed with cancer will benefit from adjuvant therapy comprising: a) determining a total level of expression of cyclin D1 within a nuclear compartment and non-nuclear compartments combined in cells of interest in a tumor tissue sample from the patient; b) determining a total level of expression of signal transducer and activator of transcription-3 (STAT-3) within the nuclear compartment and the non-nuclear compartments combined in cells of interest in the tumor tissue sample from the patient; c) determining a level of expression of thyroid transcription factor-1 (TTF1) in the nuclear compartment in cells of interest in the tumor tissue sample from the patient; d) determining a level of expression of beta-catenin in a cytoplasmic compartment in cells of interest in the tumor tissue sample from the patient; e) calculating a score based on the levels of expression determined in steps (a), (b) and (c) by: (i) adding the level of expression in step (a) with the level of expression obtained in step (b); (ii) subtracting the level of expression obtained in step (c) from the sum obtained in step (i); (iii) subtracting the level of expression obtained in step (d) from the difference obtained in step (ii); and f) correlating the score obtained in step (b) with a series of predetermined reference scores associated with a series of high risk and low risk patients; so as to thereby determine the likelihood that the patient will benefit from adjuvant therapy.

The invention provides for a kit comprising at least three of the following: a first stain specific for thyroid transcription factor-(TTF1); a second stain specific for signal transducer and activator of transcription-3 (STAT-3); a third stain specific for beta-catenin; a fourth stain specific for cyclin D1; and instructions for using the kit.

The invention provides for a non-transitory computer readable medium having program code recorded thereon that, when executed on a computing system, automatically processes data, the program code comprising: code for processing a digital microscopy image of a stained tumor specimen taken from a cancer patient to extract data related to intensity values associated with one or more stains; code for processing the extracted data to arrive at a value for intensity per pixel for each of the one or more stains; code for processing pixel intensity of at least one stain for determining pixels associated with a preselected subcompartment and determining the area of the subcompartment for use as a denominator; code for processing pixel intensity of a second stain for determining an expression level of a biomarker and a value for total biomarker intensity in the same preselected subcompartment for use as a numerator; code for calculating from the numerator and denominator a score of the biomarker expression per area; code for collecting the score of each of at least three of the following four biomarkers, including thyroid transcription factor-1 (TTF1), signal transducer and activator of transcription-3 (STAT-3), beta-catenin and cyclin D1; and code for incorporating the scores of the at least three biomarkers into a score model for arriving at a prognostic score.

A method for making a prognosis for a patient having a tumor associated with adenocarcimona of the lung which comprises: a) measuring in a sample of the patient's tumor a level of expression for each of at least three biomarkers selected from the following group: thyroid transcription factor-1 (TTF1), signal transducer and activator of transcription-3 (STAT-3), beta-catenin and cyclin D1; b) calculating a score based on the level of expression measured in step (a) for each of the biomarkers; and c) correlating the score obtained in step (b) with a series of predetermined reference scores associated with a series of prognoses; so as to thereby make a prognosis for the patient.

A method for identifying a patient having a tumor associated with adenocarcimona of the lung as having a 40% or less chance of survival after five years if treated only by surgery comprising: a) measuring in a sample of the patient's tumor a level of expression for at least three biomarkers selected from the following group: thyroid transcription factor-1 (TTF1), signal transducer and activator of transcription-3 (STAT-3), beta-catenin and cyclin D1; b) calculating a score based on the levels of expression measured in step (a); and c) comparing the score obtained in step (b) with a predetermined reference score associated with probability of survival after five years if treated only by surgery; wherein if the score obtained in step (b) is greater than the predetermined reference score the patient has a 40% or less chance of survival after five years if treated only by surgery.

A method for determining the likelihood that a patient having a tumor associated with adenocarcinoma of the lung will not survive after five years if treated only by surgery: a) measuring in a sample of the patient's tumor a level of expression for at each of least three biomarkers selected from the following group: thyroid transcription factor-1 (TTF1), signal transducer and activator of transcription-3 (STAT-3), beta-catenin and cyclin D1; b) calculating a score based on the levels of expression measured in step (a); and c) correlating the score obtained in step (b) with a series of predetermined reference scores associated with survival; so as to thereby determine the likelihood that the patient will not survive after five years if treated only by surgery.

A method for determining the likelihood that a patient having a tumor associated with adenocarcinoma of the lung will have a higher chance of survival after surgery from adjuvant therapy in addition to the surgery comprising: a) measuring in a sample of the patient's tumor a level of expression for each of at least three biomarkers selected from the following group: thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with survival; so as to thereby determine the likelihood that the patient will have a higher chance of survival after surgery from adjuvant therapy in addition to surgery.

A method for determining the likelihood that a patient having a tumor associated with adenocarcinoma of the lung will have a higher chance of survival after surgery from adjuvant therapy in addition to the surgery comprising: a) measuring in a sample of the patient's tumor a level of expression for each of thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with survival; so as to thereby determine the likelihood that the patient will have a higher chance of survival after surgery from adjuvant therapy in addition to surgery.

A method for determining the likelihood that a patient having a tumor associated with adenocarcinoma of the lung will have a higher chance of survival after surgery from adjuvant therapy in addition to the surgery comprising: a) measuring a total level of expression of cyclin D1 within a nuclear compartment and non-nuclear compartments combined in cells of interest in a tumor sample from the patient; b) measuring a total level of expression of signal transducer and activator of transcription-3 (STAT-3) within the nuclear compartment and the non-nuclear compartments combined in cells of interest in the sample from the patient; c) measuring a level of expression of thyroid transcription factor-1 (TTF1) in the nuclear compartment in cells of interest in the sample from the patient; d) measuring a level of expression of beta-catenin in a cytoplasmic compartment in cells of interest in the sample from the patient; e) calculating a score based on the levels of expression determined in steps (a) through (d); and f) correlating the score obtained in step (b) with a series of predetermined reference scores associated with survival; so as to thereby determine the likelihood that the patient will have a higher chance of survival after surgery from adjuvant therapy in addition to surgery.

A method for determining the likelihood that a patient having a tumor associated with adenocarcinoma of the lung will have a higher chance of survival after surgery from adjuvant therapy in addition to the surgery comprising: a) measuring a total level of expression of cyclin D1 within a nuclear compartment and non-nuclear compartments combined in cells of interest in a tumor tissue sample from the patient; b) measuring a total level of expression of signal transducer and activator of transcription-3 (STAT-3) within the nuclear compartment and the non-nuclear compartments combined in cells of interest in the tumor tissue sample from the patient; c) measuring a level of expression of thyroid transcription factor-1 (TTF1) in the nuclear compartment in cells of interest in the tumor tissue sample from the patient; d) measuring a level of expression of beta-catenin in a cytoplasmic compartment in cells of interest in the tumor tissue sample from the patient; e) calculating a score based on the levels of expression measured in steps (a), (b) and (c) by: (i) adding the level of expression in step (a) with the level of expression obtained in step (b); (ii) subtracting the level of expression obtained in step (c) from the sum obtained in step (i); (iii) subtracting the level of expression obtained in step (d) from the difference obtained in step (ii); and f) correlating the score obtained in step (b) with a series of predetermined reference scores associated with survival; so as to thereby determine the likelihood that the patient will have a higher chance of survival after surgery from adjuvant therapy in addition to surgery.

A method for determining whether a patient having a tumor associated with adenocarcinoma of the lung will have a greater probability of survival after a predetermined period of time if treated by surgery and adjunct therapy than if treated by surgery alone comprising: a) measuring in a sample of the patient's tumor a level of expression for at least three biomarkers selected from the following group: thyroid transcription factor-1 (TTF1), signal transducer and activator of transcription-3 (STAT-3), beta-catenin and cyclin D1; b) calculating a score based on the levels of expression of such at least three biomarkers measured in step (a); and c) comparing the score obtained in step (b) with a predetermined reference score associated with an increased probability of survival after such predetermined period of time if treated by surgery and adjunct therapy as compared with treatment by surgery alone; so as to determine if the patient's probability of survival would be greater if treated by surgery and adjunct therapy.

A method for determining whether a patient having a tumor associated with adenocarcinoma of the lung will have a greater probability of survival after a predetermined period of time if treated by surgery and adjunct therapy than if treated by surgery alone comprising: a) measuring in a sample of the patient's tumor a level of expression for at least three biomarkers selected from the following group: thyroid transcription factor-1 (TTF1), signal transducer and activator of transcription-3 (STAT-3), beta-catenin and cyclin D1; b) calculating a score based on the levels of expression of such at least three biomarkers measured in step (a); and c) comparing the score obtained in step (b) with a predetermined reference score associated with an increased probability of survival after such predetermined period of time if treated by surgery and adjunct therapy as compared with treatment by surgery alone; so as to determine if the patient's probability of survival would be greater if treated by surgery and adjunct therapy.

A method for determining the likelihood that a patient having a tumor associated with adenocarcinoma of the lung will have an increased chance of survival after surgery and adjunct therapy in addition to the surgery comprising: a) measuring in a sample of the patient's tumor a level of expression for at least three biomarkers selected from the following group: thyroid transcription factor-1 (TTF1), signal transducer and activator of transcription-3 (STAT-3), beta-catenin and cyclin D1; b) calculating a score based on the levels of expression of such at least three biomarkers measured in step (a); and c) comparing the score obtained in step (b) with a predetermined reference score associated with an increased probability of survival after such predetermined period of time if treated by surgery and adjunct therapy as compared with treatment by surgery alone; so as to determine if the patient's probability of survival would be greater if treated by surgery and adjunct therapy.

A method for determining the likelihood that a patient having a tumor associated with adenocarcinoma of the lung will have an increased chance of survival after surgery and adjunct therapy in addition to the surgery comprising: a) measuring in a sample of the patient's tumor a level of expression for each of at least three biomarkers selected from the following group: thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with survival of reference patients having tumors associated with adenocarcinomas of the lung; so as to thereby determine the likelihood that the patient will have a higher chance of survival after surgery from adjunct therapy in addition to surgery.

### BRIEF DESCRIPTION OF THE FIGURES

FIG. 1. Is a flow chart showing the steps used to develop the prognostic algorithm.

FIG. 2. Shows linear regression analyses (XY scatter plots with indicated Pearson's R coefficients) for determining the day-to-day reproducibility of expression scores for each of the antibodies used to detect each of the biomarkers in the final lung adenocarcinoma prognostic algorithm including TTF1 (FIG. 2A), STAT3 (FIG. 2B), Beta-catenin (FIG. 2C) and Cyclin D1 (FIG. 2D). Also, provided are Western blot results (inset) of cell lysates demonstrating specificity of the antibody for the indicated biomarkers.

FIG. 3. Representative grayscale digital images of biomarker staining in lung adenocarcinoma specimens including staining for TTF1 (FIG. 3A), STAT3 (FIG. 3B), Beta catenin (FIG. 3C) and Cyclin D1 (FIG. 3D). Expected patterns of expression are observed indicating specificity of the assay in FFPE tissue specimens (TTF: nuclear; STAT3: cytoplasmic/nuclear; Beta-catenin: membrane/cytoplasmic; and Cyclin D1: nuclear)

FIG. 4. Kaplan Meier 8-year disease specific-survival analyses with indicated log-Rank P-values for the lung adenocarcinoma prognostic scores. Prognostic scores for the training set (FIGS. 4A and 4C) were divided into 3 equal groups representing high, intermediate, and low risk and the respective cutpoints were subsequently applied to validation cohort (FIGS. 4B and 4D). Analysis was done for all adenocarcinoma patients (FIGS. 4A and 4B) and Stage 1 patients only (FIGS. 4C and 4D). There was significant prediction of survival in the training set for all patients (FIG. 4A, Log rank p=0.002) and Stage 1 patients (FIG. 4C, Log rank p=0.017). This finding was validated in the second cohort, for all patients (FIG. 4B, Log rank p=0.037) and for stage 1 pateints (FIG. 4D, Log rank p=0.006).

FIG. 5. Kaplan Meier 8-year disease specific-survival analysis with indicated log-rank P-values for the lung adenocarcinoma prognostic scores for the same validation cohort as in FIGS. 4B and D. This analysis demonstrates that the high and intermediate prognostic groups can be combined into one group (FIG. 5B; red line, N=71) while maintaining significant survival prediction (Log rank p=0.028). This is also the case for the Stage 1 (FIG. 5C, Log rank p=0.043) and Stage 1a patients (FIG. 5D; Log rank p=0.055, significant at the 10% level).

FIG. 6. Kaplan Meier 8-year disease specific-survival analysis with indicated log-rank P-values for the squamous cell lung carcinoma prognostic scores and probability of survival showing that the lung adenocarcinoma prognostic scores are not applicable to squamous cell carcinoma (log rank p=0.47).

FIG. 7. Forest plot showing mean hazard ratios and 95% confidence intervals for the training set and the validation set. Hazard ratios and 95% confidence intervals are above one indicating significant prediction of decreased overall survival. Values for both univariate risk score and risk scores adjusted for Stage (adjusted) are provided. These data represent a summary of data in Tables 5-7, discussed in the Examples section.

### DETAILED DESCRIPTION OF THE INVENTION

A “predetermined reference score cutpoint” associated with high risk and low risk patients refers to a cutpoint associated with dividing a group of patients into high risk and low risk patients.

The invention provides a method for making a prognosis for a patient afflicted with a type of cancer which comprises: a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; b) calculating a score based on the levels of expression determined in step (a); c) correlating the score obtained in step (b) with a series of predetermined reference scores associated with a series of prognoses; so as to thereby make a prognosis for the patient.

The levels of expression of all four biomarkers, thyroid transcription factor-1 (TTF1), signal transducer and activator of transcription-3 (STAT-3), beta-catenin and cyclin D1, may be determined.

The cancer may be a lung cancer.

The lung cancer may be adenocarcinoma.

The adenocarcinoma of the lung may be a stage I cancer.

The levels of expression of thyroid transcription factor-1 (TTF1), signal transducer and activator of transcription-3 (STAT-3), beta-catenin and cyclin D1 may determined using an automated pathology system.

The levels of expression of thyroid transcription factor-1 (TTF1), signal transducer and activator of transcription-3 (STAT-3), beta-catenin and cyclin D1 may be determined using a quantitative image analysis procedure.

Numerous quantitative image analysis procedures are known in the art. An example of a quantitative image analysis procedure that may be used to determine the levels of expression include AQUA® technology procedures, as described in issued U.S. Pat. No. 7,219,016, U.S. Pat. No. 8,036,833 and U.S. Pat. No. 8,121,794, which are incorporated by reference in to this application in its entirety.

Other quantitative image analysis procedures may include the Bliss system, the ACIS system, the IVision and GenoMx system, the ScanScope Systems, the Ariol SL-50 System, the Vectra and Nuance systems, Leica microscope systems, and the LSC system which are available from the following respective manufacturers: Bacus Laboratories, Inc., Clarient, Inc., BioGenex, DakoCytomation, Applied Imaging Corporation, Perkin Elmer (Caliper), Leica and CompuCyte Corporation (for more information, please see Immunohistochemistry and Quantitative Analysis of Protein Expression, by Melissa Cregger, Aaron J. Berger, and David L. Rimm, published July 2006 in Archives of Pathology and Laboratory Medicine); the procedure described in The Relative Distribution of Membranous and Cytoplasmic Met is a Prognostic Indicator in Stage I and II Colon Cancer, by Fiora Ginty, Sudeshna Adak, Ali Can, Michael Gerdes, Melinda Larsen, h arvey Cline, Robert Filkins, Zhengyu Pang, Qing Li, and Michael C. Montalto, published Jun. 15, 2008 in Clinical Cancer Research; and the procedure described in Quantitative Fluorescence Imaging Analysis for Cancer Biomarker Discovery: Applications to β-Catenin in Archives Prostate Specimens, by Dali Huang, George P. Casale, Jun Tian, Nizar K. Wehbi, Neil A. Abrahams, Zahid Kaleem, Lynette M. Smith, Sonny L. Johansson, Johny E. Elkahwaji, and George P. Hemstreet III published July 2007 in Cancer Epidemiology Biomarkers). The disclosures of these publications is hereby incorporated by reference into this application.

As used in this application, the term sample can refer to various kinds of sample, for example, a tissue sample and a cytology specimen.

The tissue sample may be a fixed tissue section.

The method of this invention may be used in formalin-fixed paraffin-embedded sections, fine needle aspirate and other histological sample types.

The method of this invention may be applied to tumor material obtained by surgical biopsy, bronchoscopic biopsies and fine-needle aspirations.

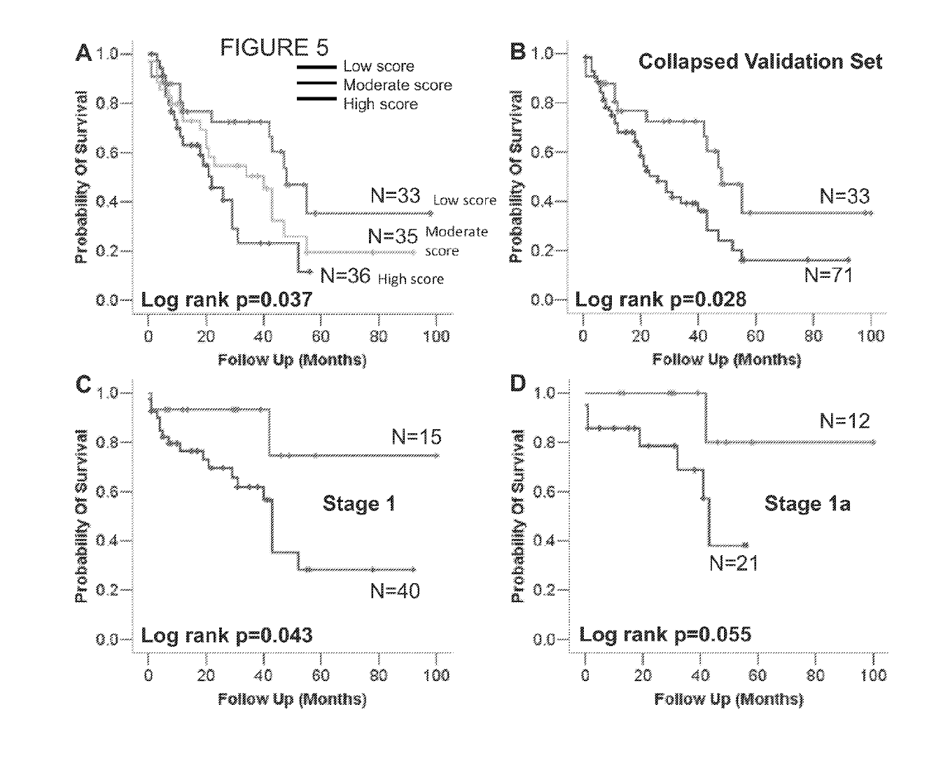

A patient may be considered relatively high risk if the patient has a 40% or less chance of survival at five years. Alternatively a patient may be considered at relatively high risk if the patient has a survival profile such as those shown in FIG. 5, with a high score (lower curve).

A patient may be considered relatively low risk if the patient has a 95% or more chance of survival at five years. Alternatively a patient may be considered at relatively low risk if the patient has a survival profile such as those shown in FIG. 5, with a low score (upper curve)

The invention provides a method for classifying a patient diagnosed with cancer as being at a low risk for a recurrence of cancer comprising: a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; b) calculating a score based on the levels of expression determined in step (a); and c) comparing the score obtained in step (b) with a predetermined reference score cutpoint separating high risk from low risk patients; wherein the patient is at a low risk of developing a recurrence of cancer if the score obtained in step (b) is less than the predetermined reference score cutpoint.

The levels of expression of all four biomarkers, thyroid transcription factor-1 (TTF1), signal transducer and activator of transcription-3 (STAT-3), beta-catenin and cyclin D1, may be determined.

The cancer may be a lung cancer.

The lung cancer may be adenocarcinoma.

The adenocarcinoma of the lung may be a stage I cancer.

The levels of expression of thyroid transcription factor-1 (TTF1), signal transducer and activator of transcription-3 (STAT-3), beta-catenin and cyclin D1 may determined using an automated pathology system.

The levels of expression of thyroid transcription factor-1 (TTF1), signal transducer and activator of transcription-3 (STAT-3), beta-catenin and cyclin D1 may be determined using a quantitative image analysis procedure.

Numerous quantitative image analysis procedures are known in the art as described above.

Other quantitative image analysis procedures can be used such as those described above.

As used in this application, the term sample can refer to various kinds of sample, for example, a tissue sample and a cytology specimen.

The tissue sample may be a fixed tissue section.

The method of this invention may be used in formalin-fixed paraffin-embedded sections, fine needle aspirate and other histological sample types.

The method of this invention may be applied to tumor material obtained by surgical biopsy, bronchoscopic biopsies and fine-needle aspirations.

A patient may be considered high risk if the patient has a 40% chance of survival at five years.

A patient may be considered low risk if the patient has a 95% chance of survival at five years.

The levels of expression of all four biomarkers, thyroid transcription factor-1 (TTF1), signal transducer and activator of transcription-3 (STAT-3), beta-catenin and cyclin D1, may be determined.

The cancer may be a lung cancer.

The lung cancer may be adenocarcinoma.

The adenocarcinoma of the lung may be a stage I cancer.

The levels of expression of thyroid transcription factor-1 (TTF1), signal transducer and activator of transcription-3 (STAT-3), beta-catenin and cyclin D1 may determined using an automated pathology system.

The levels of expression of thyroid transcription factor-1 (TTF1), signal transducer and activator of transcription-3 (STAT-3), beta-catenin and cyclin D1 may be determined using a quantitative image analysis procedure.

Numerous quantitative image analysis procedures are known in the art as described above.

Other quantitative image analysis procedures can be used such as those described above.

As used in this application, the term sample can refer to various kinds of sample, for example, a tissue sample and a cytology specimen.

The tissue sample may be a fixed tissue section.

The method of this invention may be used in formalin-fixed paraffin-embedded sections, fine needle aspirate and other histological sample types.

The method of this invention may be applied to tumor material obtained by surgical biopsy, bronchoscopic biopsies and fine-needle aspirations.

A patient may be considered high risk if the patient has a 40% chance of survival at five years.

A patient may be considered low risk if the patient has a 95% chance of survival at five years.

The invention provides a method for classifying a patient diagnosed with cancer as being at a high risk for a recurrence of cancer comprising: a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; b) calculating a score based on the levels of expression determined in step (a); and c) comparing the score obtained in step (b) with a predetermined reference score cutpoint separating high risk from low risk patients; wherein the patient is at a high risk of developing a recurrence of cancer if the score obtained in step (b) is greater than the predetermined reference score cutpoint.

The levels of expression of all four biomarkers, thyroid transcription factor-1 (TTF1), signal transducer and activator of transcription-3 (STAT-3), beta-catenin and cyclin D1, may be determined.

The cancer may be a lung cancer.

The lung cancer may be adenocarcinoma.

The adenocarcinoma of the lung may be a stage I cancer.

The levels of expression of thyroid transcription factor-1 (TTF1), signal transducer and activator of transcription-3 (STAT-3), beta-catenin and cyclin D1 may determined using an automated pathology system.

The levels of expression of thyroid transcription factor-1 (TTF1), signal transducer and activator of transcription-3 (STAT-3), beta-catenin and cyclin D1 may be determined using a quantitative image analysis procedure.

Numerous quantitative image analysis procedures are known in the art as described above.

Other quantitative image analysis procedures can be used such as those described above.

As used in this application, the term sample can refer to various kinds of sample, for example, a tissue sample and a cytology specimen.

The tissue sample may be a fixed tissue section.

The method of this invention may be used in formalin-fixed paraffin-embedded sections, fine needle aspirate and other histological sample types.

The method of this invention may be applied to tumor material obtained by surgical biopsy, bronchoscopic biopsies and fine-needle aspirations.

A patient may be considered high risk if the patient has a 40% chance of survival at five years.

A patient may be considered low risk if the patient has a 95% chance of survival at five years.

A method for determining the likelihood that a patient diagnosed with cancer will develop a recurrence of cancer comprising: a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with high risk and low risk patients; so as to thereby determine the likelihood that the patient will develop a recurrence of cancer.

The series of predetermined reference scores may be used to generate a predetermined reference score cutpoint separating high risk from low risk patients wherein there is a likelihood that the patient will benefit from adjuvant therapy if the score obtained in step (b) is greater than the predetermined reference score cutpoint.

The levels of expression of all four biomarkers, thyroid transcription factor-1 (TTF1), signal transducer and activator of transcription-3 (STAT-3), beta-catenin and cyclin D1, may be determined.

The cancer may be a lung cancer.

The lung cancer may be adenocarcinoma.

The adenocarcinoma of the lung may be a stage I cancer.

The levels of expression of thyroid transcription factor-1 (TTF1), signal transducer and activator of transcription-3 (STAT-3), beta-catenin and cyclin D1 may determined using an automated pathology system.

The levels of expression of thyroid transcription factor-1 (TTF1), signal transducer and activator of transcription-3 (STAT-3), beta-catenin and cyclin D1 may be determined using a quantitative image analysis procedure.

Numerous quantitative image analysis procedures are known in the art as described above.

Other quantitative image analysis procedures can be used such as those described above.

As used in this application, the term sample can refer to various kinds of sample, for example, a tissue sample and a cytology specimen.

The tissue sample may be a fixed tissue section.

The method of this invention may be used in formalin-fixed paraffin-embedded sections, fine needle aspirate and other histological sample types.

The method of this invention may be applied to tumor material obtained by surgical biopsy, bronchoscopic biopsies and fine-needle aspirations.

A patient may be considered high risk if the patient has a 40% chance of survival at five years.

A patient may be considered low risk if the patient has a 95% chance of survival at five years.

A method for determining the likelihood that a patient diagnosed with cancer will benefit from adjuvant therapy comprising: a) determining in a sample of a tumor from the patient a level of expression for at least three biomarkers from the following group: thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with a series of high risk and low risk patients; so as to thereby determine the likelihood that the patient will benefit from adjuvant therapy.

The series of predetermined reference scores may be used to generate a predetermined reference score cutpoint separating high risk from low risk patients wherein there is a likelihood that the patient will benefit from adjuvant therapy if the score obtained in step (b) is greater than the predetermined reference score cutpoint.

The levels of expression of all four biomarkers, thyroid transcription factor-1 (TTF1), signal transducer and activator of transcription-3 (STAT-3), beta-catenin and cyclin D1, may be determined.

The cancer may be a lung cancer.

The lung cancer may be adenocarcinoma.

The adenocarcinoma of the lung may be a stage I cancer.

The adjuvant therapy may be chemotherapy.

The therapeutic treatments may include erlotinib, gefitinib, bevacizumab, sorafenib, docetaxel, gemcitabine, pemetrexed, and cisplatin.

The levels of expression of thyroid transcription factor-1 (TTF1), signal transducer and activator of transcription-3 (STAT-3), beta-catenin and cyclin D1 may determined using an automated pathology system.

The levels of expression of thyroid transcription factor-1 (TTF1), signal transducer and activator of transcription-3 (STAT-3), beta-catenin and cyclin D1 may be determined using a quantitative image analysis procedure.

Numerous quantitative image analysis procedures are known in the art as described above.

Other quantitative image analysis procedures can be used such as those described above.

As used in this application, the term sample can refer to various kinds of sample, for example, a tissue sample and a cytology specimen.

The tissue sample may be a fixed tissue section.

The method of this invention may be used in formalin-fixed paraffin-embedded sections, fine needle aspirate and other histological sample types.

The method of this invention may be applied to tumor material obtained by surgical biopsy, bronchoscopic biopsies and fine-needle aspirations.

A patient may be considered high risk if the patient has a 40% chance of survival at five years.

A patient may be considered low risk if the patient has a 95% chance of survival at five years.

A method for determining the likelihood that a patient diagnosed with cancer will benefit from adjuvant therapy comprising: a) determining in a sample of a tumor from the patient a level of expression for thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with a series of high risk and low risk patients; so as to thereby determine the likelihood that the patient will benefit from adjuvant therapy.

The series of predetermined reference scores may be used to generate a predetermined reference score cutpoint separating high risk from low risk patients wherein there is a likelihood that the patient will benefit from adjuvant therapy if the score obtained in step (b) is greater than the predetermined reference score cutpoint.

The cancer may be a lung cancer.

The lung cancer may be adenocarcinoma.

The adenocarcinoma of the lung may be a stage I cancer.

The adjuvant therapy may be chemotherapy.

The therapeutic treatments may include erlotinib, gefitinib, bevacizumab, sorafenib, docetaxel, gemcitabine, pemetrexed, and cisplatin.

The levels of expression of thyroid transcription factor-1 (TTF1), signal transducer and activator of transcription-3 (STAT-3), beta-catenin and cyclin D1 may determined using an automated pathology system.

The levels of expression of thyroid transcription factor-1 (TTF1), signal transducer and activator of transcription-3 (STAT-3), beta-catenin and cyclin D1 may be determined using a quantitative image analysis procedure.

Numerous quantitative image analysis procedures are known in the art as described above.

Other quantitative image analysis procedures can be used such as those described above.

As used in this application, the term sample can refer to various kinds of sample, for example, a tissue sample and a cytology specimen.

The tissue sample may be a fixed tissue section.

The method of this invention may be used in formalin-fixed paraffin-embedded sections, fine needle aspirate and other histological sample types.

The method of this invention may be applied to tumor material obtained by surgical biopsy, bronchoscopic biopsies and fine-needle aspirations.

A patient may be considered high risk if the patient has a 40% chance of survival at five years.

A patient may be considered low risk if the patient has a 95% chance of survival at five years.

A method for determining the likelihood that a patient diagnosed with cancer will benefit from adjuvant therapy comprising: a) determining a total level of expression of cyclin D1 within a nuclear compartment and non-nuclear compartments combined in cells of interest in a tumor sample from the patient; b) determining a total level of expression of signal transducer and activator of transcription-3 (STAT-3) within the nuclear compartment and the non-nuclear compartments combined in cells of interest in the sample from the patient; c) determining a level of expression of thyroid transcription factor-1 (TTF1) in the nuclear compartment in cells of interest in the sample from the patient; d) determining a level of expression of beta-catenin in a cytoplasmic compartment in cells of interest in the sample from the patient; e) calculating a score based on the levels of expression determined in steps (a) through (d); and f) correlating the score obtained in step (b) with a series of predetermined reference scores associated with a series of high risk and low risk patients; so as to thereby determine the likelihood that the patient will benefit from adjuvant therapy.

The series of predetermined reference scores may be used to generate a predetermined reference score cutpoint separating high risk from low risk patients wherein there is a likelihood that the patient will benefit from adjuvant therapy if the score obtained in step (b) is greater than the predetermined reference score cutpoint.

The cancer may be a lung cancer.

The lung cancer may be adenocarcinoma.

The adenocarcinoma of the lung may be a stage I cancer.

The adjuvant therapy may be chemotherapy.

The therapeutic treatments may include erlotinib, gefitinib, bevacizumab, sorafenib, docetaxel, gemcitabine, pemetrexed, and cisplatin.

The levels of expression of thyroid transcription factor-1 (TTF1), signal transducer and activator of transcription-3 (STAT-3), beta-catenin and cyclin D1 may determined using an automated pathology system.

The levels of expression of thyroid transcription factor-1 (TTF1), signal transducer and activator of transcription-3 (STAT-3), beta-catenin and cyclin D1 may be determined using a quantitative image analysis procedure.

Numerous quantitative image analysis procedures are known in the art as described above.

Other quantitative image analysis procedures can be used such as those described above.

As used in this application, the term sample can refer to various kinds of sample, for example, a tissue sample and a cytology specimen.

The tissue sample may be a fixed tissue section.

The method of this invention may be used in formalin-fixed paraffin-embedded sections, fine needle aspirate and other histological sample types.

The method of this invention may be applied to tumor material obtained by surgical biopsy, bronchoscopic biopsies and fine-needle aspirations.

A patient may be considered high risk if the patient has a 40% chance of survival at five years.

A patient may be considered low risk if the patient has a 95% chance of survival at five years.

A method for determining the likelihood that a patient diagnosed with cancer will benefit from adjuvant therapy comprising: a) determining a total level of expression of cyclin D1 within a nuclear compartment and non-nuclear compartments combined in cells of interest in a tumor tissue sample from the patient; b) determining a total level of expression of signal transducer and activator of transcription-3 (STAT-3) within the nuclear compartment and the non-nuclear compartments combined in cells of interest in the tumor tissue sample from the patient; c) determining a level of expression of thyroid transcription factor-1 (TTF1) in the nuclear compartment in cells of interest in the tumor tissue sample from the patient; d) determining a level of expression of beta-catenin in a cytoplasmic compartment in cells of interest in the tumor tissue sample from the patient; e) calculating a score based on the levels of expression determined in steps (a), (b) and (c) by: (i) adding the level of expression in step (a) with the level of expression obtained in step (b); (ii) subtracting the level of expression obtained in step (c) from the sum obtained in step (i); (iii) subtracting the level of expression obtained in step (d) from the difference obtained in step (ii); and f) correlating the score obtained in step (b) with a series of predetermined reference scores associated with a series of high risk and low risk patients; so as to thereby determine the likelihood that the patient will benefit from adjuvant therapy.

The series of predetermined reference scores may be used to generate a predetermined reference score cutpoint separating high risk from low risk patients wherein there is a likelihood that the patient will benefit from adjuvant therapy if the score obtained in step (b) is greater than the predetermined reference score cutpoint.

The cancer may be a lung cancer.

The lung cancer may be adenocarcinoma.

The adenocarcinoma of the lung may be a stage I cancer.

The adjuvant therapy may be chemotherapy.

The therapeutic treatments may include erlotinib, gefitinib, bevacizumab, sorafenib, docetaxel, gemcitabine, pemetrexed, and cisplatin.

The levels of expression of thyroid transcription factor-1 (TTF1), signal transducer and activator of transcription-3 (STAT-3), beta-catenin and cyclin D1 may determined using an automated pathology system.

The levels of expression of thyroid transcription factor-1 (TTF1), signal transducer and activator of transcription-3 (STAT-3), beta-catenin and cyclin D1 may be determined using a quantitative image analysis procedure.

Numerous quantitative image analysis procedures are known in the art as described above.

Other quantitative image analysis procedures can be used such as those described above.

As used in this application, the term sample can refer to various kinds of sample, for example, a tissue sample and a cytology specimen.

The tissue sample may be a fixed tissue section.

The method of this invention may be used in formalin-fixed paraffin-embedded sections, fine needle aspirate and other histological sample types.

The method of this invention may be applied to tumor material obtained by surgical biopsy, bronchoscopic biopsies and fine-needle aspirations.

A patient may be considered high risk if the patient has a 40% chance of survival at five years.

A patient may be considered low risk if the patient has a 95% chance of survival at five years.

The invention provides for a kit comprising at least three of the following: a first stain specific for thyroid transcription factor-(TTF1); a second stain specific for signal transducer and activator of transcription-3 (STAT-3); a third stain specific for beta-catenin; a fourth stain specific for cyclin D1; and instructions for using the kit.

The kit may further comprise predetermined reference score cutpoints associated with high risk patients and with low risk patients.

The invention provides for a non-transitory computer readable medium having program code recorded thereon that, when executed on a computing system, automatically processes data, the program code comprising: code for processing a digital microscopy image of a stained tumor specimen taken from a cancer patient to extract data related to intensity values associated with one or more stains; code for processing the extracted data to arrive at a value for intensity per pixel for each of the one or more stains; code for processing pixel intensity of at least one stain for determining pixels associated with a preselected subcompartment and determining the area of the subcompartment for use as a denominator; code for processing pixel intensity of a second stain for determining an expression level of a biomarker and a value for total biomarker intensity in the same preselected subcompartment for use as a numerator; code for calculating from the numerator and denominator a score of the biomarker expression per area; code for collecting the score of each of at least three of the following four biomarkers, including thyroid transcription factor-1 (TTF1), signal transducer and activator of transcription-3 (STAT-3), beta-catenin and cyclin D1; and code for incorporating the scores of the at least three biomarkers into a score model for arriving at a prognostic score.

A method for making a prognosis for a patient having a tumor associated with adenocarcimona of the lung which comprises: a) measuring in a sample of the patient's tumor a level of expression for each of at least three biomarkers selected from the following group: thyroid transcription factor-1 (TTF1), signal transducer and activator of transcription-3 (STAT-3), beta-catenin and cyclin D1; b) calculating a score based on the level of expression measured in step (a) for each of the biomarkers; and c) correlating the score obtained in step (b) with a series of predetermined reference scores associated with a series of prognoses; so as to thereby make a prognosis for the patient.

A method for identifying a patient having a tumor associated with adenocarcimona of the lung as having a 40% or less chance of survival after five years if treated only by surgery comprising: a) measuring in a sample of the patient's tumor a level of expression for at least three biomarkers selected from the following group: thyroid transcription factor-1 (TTF1), signal transducer and activator of transcription-3 (STAT-3), beta-catenin and cyclin D1; b) calculating a score based on the levels of expression measured in step (a); and c) comparing the score obtained in step (b) with a predetermined reference score associated with probability of survival after five years if treated only by surgery; wherein if the score obtained in step (b) is greater than the predetermined reference score the patient has a 40% or less chance of survival after five years if treated only by surgery.

A method for determining the likelihood that a patient having a tumor associated with adenocarcinoma of the lung will not survive after five years if treated only by surgery: a) measuring in a sample of the patient's tumor a level of expression for at each of least three biomarkers selected from the following group: thyroid transcription factor-1 (TTF1), signal transducer and activator of transcription-3 (STAT-3), beta-catenin and cyclin D1; b) calculating a score based on the levels of expression measured in step (a); and c) correlating the score obtained in step (b) with a series of predetermined reference scores associated with surival; so as to thereby determine the likelihood that the patient will not survive after five years if treated only by surgery.

A method for determining the likelihood that a patient having a tumor associated with adenocarcinoma of the lung will have a higher chance of survival after surgery from adjuvant therapy in addition to the surgery comprising: a) measuring in a sample of the patient's tumor a level of expression for each of at least three biomarkers selected from the following group: thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with survival; so as to thereby determine the likelihood that the patient will have a higher chance of survival after surgery from adjuvant therapy in addition to surgery.

A method for determining the likelihood that a patient having a tumor associated with adenocarcinoma of the lung will have a higher chance of survival after surgery from adjuvant therapy in addition to the surgery comprising: a) measuring in a sample of the patient's tumor a level of expression for each of thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with survival; so as to thereby determine the likelihood that the patient will have a higher chance of survival after surgery from adjuvant therapy in addition to surgery.

A method for determining the likelihood that a patient having a tumor associated with adenocarcinoma of the lung will have a higher chance of survival after surgery from adjuvant therapy in addition to the surgery comprising: a) measuring a total level of expression of cyclin D1 within a nuclear compartment and non-nuclear compartments combined in cells of interest in a tumor sample from the patient; b) measuring a total level of expression of signal transducer and activator of transcription-3 (STAT-3) within the nuclear compartment and the non-nuclear compartments combined in cells of interest in the sample from the patient; c) measuring a level of expression of thyroid transcription factor-1 (TTF1) in the nuclear compartment in cells of interest in the sample from the patient; d) measuring a level of expression of beta-catenin in a cytoplasmic compartment in cells of interest in the sample from the patient; e) calculating a score based on the levels of expression determined in steps (a) through (d); and f) correlating the score obtained in step (b) with a series of predetermined reference scores associated with survival; so as to thereby determine the likelihood that the patient will have a higher chance of survival after surgery from adjuvant therapy in addition to surgery.

A method for determining the likelihood that a patient having a tumor associated with adenocarcinoma of the lung will have a higher chance of survival after surgery from adjuvant therapy in addition to the surgery comprising: a) measuring a total level of expression of cyclin D1 within a nuclear compartment and non-nuclear compartments combined in cells of interest in a tumor tissue sample from the patient; b) measuring a total level of expression of signal transducer and activator of transcription-3 (STAT-3) within the nuclear compartment and the non-nuclear compartments combined in cells of interest in the tumor tissue sample from the patient; c) measuring a level of expression of thyroid transcription factor-1 (TTF1) in the nuclear compartment in cells of interest in the tumor tissue sample from the patient; d) measuring a level of expression of beta-catenin in a cytoplasmic compartment in cells of interest in the tumor tissue sample from the patient; e) calculating a score based on the levels of expression measured in steps (a), (b) and (c) by: (i) adding the level of expression in step (a) with the level of expression obtained in step (b); (ii) subtracting the level of expression obtained in step (c) from the sum obtained in step (i); (iii) subtracting the level of expression obtained in step (d) from the difference obtained in step (ii); and f) correlating the score obtained in step (b) with a series of predetermined reference scores associated with survival; so as to thereby determine the likelihood that the patient will have a higher chance of survival after surgery from adjuvant therapy in addition to surgery.

The series of predetermined reference scores may be used to generate a predetermined reference score associated with survival wherein there is a likelihood that the patient will not survive after five years if treated only by surgery if the score obtained in step (b) is greater than the predetermined reference score.

A method for determining whether a patient having a tumor associated with adenocarcinoma of the lung will have a greater probability of survival after a predetermined period of time if treated by surgery and adjunct therapy than if treated by surgery alone comprising: a) measuring in a sample of the patient's tumor a level of expression for at least three biomarkers selected from the following group: thyroid transcription factor-1 (TTF1), signal transducer and activator of transcription-3 (STAT-3), beta-catenin and cyclin D1; b) calculating a score based on the levels of expression of such at least three biomarkers measured in step (a); and c) comparing the score obtained in step (b) with a predetermined reference score associated with an increased probability of survival after such predetermined period of time if treated by surgery and adjunct therapy as compared with treatment by surgery alone; so as to determine if the patient's probability of survival would be greater if treated by surgery and adjunct therapy.

A method for determining whether a patient having a tumor associated with adenocarcinoma of the lung will have a greater probability of survival after a predetermined period of time if treated by surgery and adjunct therapy than if treated by surgery alone comprising: a) measuring in a sample of the patient's tumor a level of expression for at least three biomarkers selected from the following group: thyroid transcription factor-1 (TTF1), signal transducer and activator of transcription-3 (STAT-3), beta-catenin and cyclin D1; b) calculating a score based on the levels of expression of such at least three biomarkers measured in step (a); and c) comparing the score obtained in step (b) with a predetermined reference score associated with an increased probability of survival after such predetermined period of time if treated by surgery and adjunct therapy as compared with treatment by surgery alone; so as to determine if the patient's probability of survival would be greater if treated by surgery and adjunct therapy.

A method for determining the likelihood that a patient having a tumor associated with adenocarcinoma of the lung will have an increased chance of survival after surgery and adjunct therapy in addition to the surgery comprising: a) measuring in a sample of the patient's tumor a level of expression for at least three biomarkers selected from the following group: thyroid transcription factor-1 (TTF1), signal transducer and activator of transcription-3 (STAT-3), beta-catenin and cyclin D1; b) calculating a score based on the levels of expression of such at least three biomarkers measured in step (a); and c) comparing the score obtained in step (b) with a predetermined reference score associated with an increased probability of survival after such predetermined period of time if treated by surgery and adjunct therapy as compared with treatment by surgery alone; so as to determine if the patient's probability of survival would be greater if treated by surgery and adjunct therapy.

A method for determining the likelihood that a patient having a tumor associated with adenocarcinoma of the lung will have an increased chance of survival after surgery and adjunct therapy in addition to the surgery comprising: a) measuring in a sample of the patient's tumor a level of expression for each of at least three biomarkers selected from the following group: thyroid transcription factor-1 (TTF1), signal transducer and activator of transcription-3 (STAT-3), beta-catenin and cyclin D1; b) calculating a score based on the levels of expression determined in step (a); and c) correlating the score obtained in step (b) with a series of predetermined reference scores associated with survival of reference patients having tumors associated with adenocarcinomas of the lung; so as to thereby determine the likelihood that the patient will have a higher chance of survival after surgery from adjunct therapy in addition to surgery.

The present invention provides, among other things, methods for determining the prognosis for a patient diagnosed with cancer and the likelihood that a patient diagnosed with cancer will benefit from adjuvant therapy. While specific embodiments of the subject invention have been discussed, the specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

The following Experimental Details are set forth to aid in an understanding of the subject matter of this disclosure, but are not intended to, and should not be construed to, limit in any way the claims which follow thereafter.

### Experimental Details

### Synopsis

A critical clinical problem in management of adenocarcinoma of the lung is to determine which patients are cured by surgery alone, versus which will benefit from adjuvant therapy such as chemotherapy. In particular it is critically important to determine which low stage patients are cured by surgery alone, vs. those which will benefit from adjuvant therapy such as chemotherapy. While there are only 20% of all lung cancer patients diagnosed at low stage, that still represents over 30,000 patients in the United States.

This invention is a protein-based test that can stratify lung adenocarcinoma patients, including specifically low stage lung adenocarcinoma patients, into groups are likely to, or not likely to benefit from additional therapeutic treatment such as chemotherapy.

The new process assesses the level of four (a) key proteins using, for example, AQUA technology method of standardized quantitative immunofluorescence as previously described (Camp et al 2002 Nature Medicine 8(11)1323-1327, U.S. Pat. No. 7,219,016; Gustavson et al AQUA@ Technology and Molecular Pathology in Pathology in Drug Discovery and Development, Platero ed. John Wiley & Sons, Inc, Hoboken, N.J. 2009). The method may be used on formalin-fixed, paraffin-embedded tumor specimens, fine-needle aspirates, or other histological samples.

The four proteins that are measured are:

Thyroid Transcription Factor-1 (TTF1), Signal transducer and activator of transcription-3 (STATS), Beta-Catenin, and Cyclin D1.

Patients can then be divided into either high risk or low risk groups based on cut-points determined from previous studies. Stage 1 lung cancer patients with adenocarcinoma in the low risk group have a 95% chance of survival at 5 years, compared to a 40% chance in the high risk group. Although stage I patients are not usually given chemotherapy or other therapeutic treatment, this study would select a subset (as many as 66% of patients) that fall into the high risk group that would then benefit from additional therapeutic treatment such as chemotherapy.

In a particular embodiment, the protein biomarkers are measured in a quantitative manner using standard curves to assure accuracy and precision in measurement. From these data the risk score is then calculated using the following equation:

1.322*CyclinD1(tm)+1.0126*Stat3(tm)−1.2131*TTF1(n)−0.7748*beta-Catenin(c).

Where (tm) (n) and (c) indicate the subcellular compartment used for measuring each variable using the AQUA technology (tm=total under the tumor mask, n=nuclear, c=cytoplasmic).

It would be understood by one of skill in the art that equation may be adjusted or optimized as additional cohorts of patients are analyzed. For example, the coefficients for each marker may be optimized relative to broader patient populations.

Purpose:

The importance of definitive histologic prognosticator has significantly increased as improved cancer screening methods are identifying more lung cancer patients, and at an earlier stage of disease. Here we describe the development and validation of a 4-protein classifier that is prognostic for risk of disease recurrence in lung adenocarcinomas (AC).

### Introduction

Lung cancer, predominantly non-small cell lung cancer (NSCLC), is the most common cause of death from cancer worldwide. An estimated 226,160 new cases were diagnosed in the United States in 2012. NSCLC diagnosis and histologic leads to several subclassification of disease with adenocarcinoma being the most frequent type of NSCLC. Low stage adenocarcinoma is generally treated with surgery without adjuvant therapy. However 35-50% of patients who first present with low stage disease and are treated surgically without adjuvant therapy will suffer recurrence and death due to their disease (Jenal A, et al Cancer Statistics 2010. CA Cancer J Clin 2010; 60:277-300). Therefore a prognostic test that could identify patients at risk for recurrence of disease is needed and would provide the opportunity to treat such patients more aggressively to improve outcome.

Ideally the diagnostic test should be simple to perform, avoiding methodologies that are technically challenging requiring that they be conducted in a central laboratory. The assay methodology should be standardized and have high reproducibility so that the assay can be provided in a decentralized fashion by multiple laboratories and each can expect to get concordant results. See, for example, the standardization described in Gustavson et al., Standardization of HER2 Immunohistochemistry in Breast Cancer by Automated Quantitative Analysis, published September 2009 in Arch. Pathol. Lab. Med., the disclosure of which is hereby incorporated by reference into this application.

### Materials and Methods

### Cohorts

Two cohorts of formalin-fixed paraffin-embedded primary NSCLC tumors were used for this study. A cohort of 117 adenocarcinoma patients was used as the training set and a second independent cohort of 137 adenocarcinoma patients was used as the validation set.

Both cohorts were retrospectively collected from surgical patients from Yale-New Haven Hospital (New Haven, Conn.). Demographics and details of these cohorts are shown in Table 1. The study was approved by the institutional review boards of all centers; written informed consent was obtained for each case prior to inclusion in the study.

TABLE 1
Tumor and clinical characteristics of the training
and validation adenocarcinoma cohorts

| | Training AC Cohort | | Validation AC Cohort | |
|---|---|---|---|---|
| | (n = 117) | | (n = 137) | |
| Characteristic | N | Percent | N | Percent |
| Age | | | | |
| Median (range) | 64 (33-90) | | 62 (34-84) | |
| Mean ± SE | 64 ± 1 | | 60.91 ± 0.79 | |
| Gender | | | | |
| Male | 45 | 38.5 | 115 | 84.1 |
| Female | 72 | 61.5 | 22 | 15.9 |
| Stage | | | | |
| I | 61 | 52.1 | 38 | 27.7 |
| II | 14 | 12 | 30 | 21.9 |
| III | 28 | 23.9 | 41 | 29.9 |
| IV | 14 | 12 | 28 | 20.5 |

Abbreviations: N; number, AC; adenocarcinoma, SE; standard error

### Tissue Microarrays

Tissue specimens were prepared in a tissue microarray (TMA) format: representative tumor areas were obtained from formalin fixed paraffin embedded (FFPE) specimens of the primary tumor and two 0.6 mm cores from each tumor block were arrayed in a recipient block.

### Western Blotting

Equivalent amounts of protein (15 μg) were resolved by SDS-PAGE in 4-12E bis-tris gels (150V for 1 hr) and transferred at 45V for 2 hrs to a nitrocellulose membrane. Immunoblots were probed with primary antibodies, followed by anti-rabbit or anti-mouse HRP conjugated secondary antibodies (Santa Cruz Biotechnology, Santa Cruz, Calif.) diluted 1/4000 and detected using enhanced chemiluminescence (GH Healthcare). β-tubulin (rabbit polyclonal, Cell Signaling Technology, Danvers, Mass.) immunoblotting was used to visualize the total protein loading.

### Quantitative Immunofluorescence

The arrays were deparaffinized with xylene, rehydrated and antigen-retrieved by pressure cooking for 15 minutes in 10 mM citrate (pH=6) or 10 mM Tris/1 mM EDTA buffer (pH=9) for all primary antibodies. Slides were pre-incubated with 0.3% bovine serum albumin (BSA) in 0.1M tris-buffered saline (TBS, pH=8) for 30 minutes at room temperature. Slides were then incubated with a cocktail of the primary antibody (Table 2) and a mouse monoclonal anti-human cytokeratin antibody (clone AE1/AE3, M3515, Dako, Carpinteria, Calif.) or a wide-spectrum rabbit anti-cow cytokeratin antibody (Z0622, Dako, Carpinteria, Calif.) diluted 1:100 in BSA/TBS overnight at 4° C. This was followed by an 1-hour incubation with Alexa 546-conjugated goat anti-mouse secondary antibody (A11003, Molecular Probes, Eugene, Oreg.) diluted 1:100 in rabbit EnVision reagent (K4003, Dako, Carpinteria, Calif.) or Alexa 546-conjugated goat anti-rabbit secondary antibody (A11010, Molecular Probes, Eugene, Oreg.) diluted 1:100 in mouse EnVision reagent (K4001, Dako, Carpinteria, Calif.). Cyanine 5 (Cy5) directly conjugated to tyramide (FP1117, Perkin-Elmer, Boston, Mass.) at a 1:50 dilution was used as the fluorescent chromagen for target detection. Prolong mounting medium (ProLong Gold, P36931, Molecular Probes, Eugene, Oreg.) containing 4′,6-Diamidino-2-phenylindole (DAPI) was used to identify tissue nuclei. Positive controls used are described in detail in Supplementary Table 2. Negative control sections, in which the primary antibody was omitted, were used for each immunostaining run.

### Image Collection and Analysis

Automated Quantitative Analysis (AQUA®technology) allows exact measurement of protein concentration within subcellular compartments, as described in detail elsewhere (Camp R L, Chung G G, Rimm D L: Automated subcellular localization and quantification of protein expression in tissue microarrays. Nat Med 8:1323-7, 2002).

In brief, a series of high resolution monochromatic images were captured by the PM-2000™ digital imaging microscopy instrument (HistoRx, Branford, Conn.). For each histospot, images were obtained using the signal from the DAPI, cytokeratin-Alexa 546 and target-Cy5 channel. Target proteins were measured using a channel with emission maxima above 620 nm, in order to minimize tissue autofluorescence.

Tumor was distinguished from stromal and non-stromal elements by creating an epithelial tumor “mask” from the cytokeratin signal. This created a binary mask (each pixel being either “on” or “off”) on the basis of an intensity threshold set by visual inspection of histospots. AQUA score of target proteins in the tumor mask and subcellular compartment were calculated by dividing the target compartment pixel intensities by the area of the compartment within they were measured. AQUA scores were normalized to the exposure time and bit depth at which the images were captured, allowing scores collected at different exposure times to be directly comparable. Specimens with less that 5% tumor area per spot were not included in automated quantitative analysis for not being representative of the corresponding tumor specimen.

The image collection and analysis can also be accomplished using clustering AQUA® software, as described in U.S. Patent Application Publication No. 20090034823, entitled Compartment Segregation by Pixel Characterization Using Image Data Clustering, the contents of which is hereby incorporated by reference into this application, and as described in Gustavson et al., Development of an unsupervised pixel-based clustering algorithm for compartmentalization of immunohistochemical expression using Automated Quantitative Analysis, Appl. Immunohistochemical Mol. Morphol. 2009 July; 1794):329-37, the contents of which is hereby incorporated by reference into this application. This method and system uses autoexposure and is done automatically instead of by visual inspection of histospots.

### Statistical Analysis

AQUA scores were Log 2 normalized and scores of the validation sets were further normalized for run to run variability. Missing values were tested by Little's test for missing complete at random; cases with missing values were excluded from analysis. Pearson's correlation coefficient (R) was used to assess the correlation between AQUA scores from redundant tumor cores. An R² greater than 0.4 was indicative of good inter- and intra-array reproducibility and thus the average values for all target proteins AQUA scores from duplicate samples were calculated and treated as independent continuous variables.

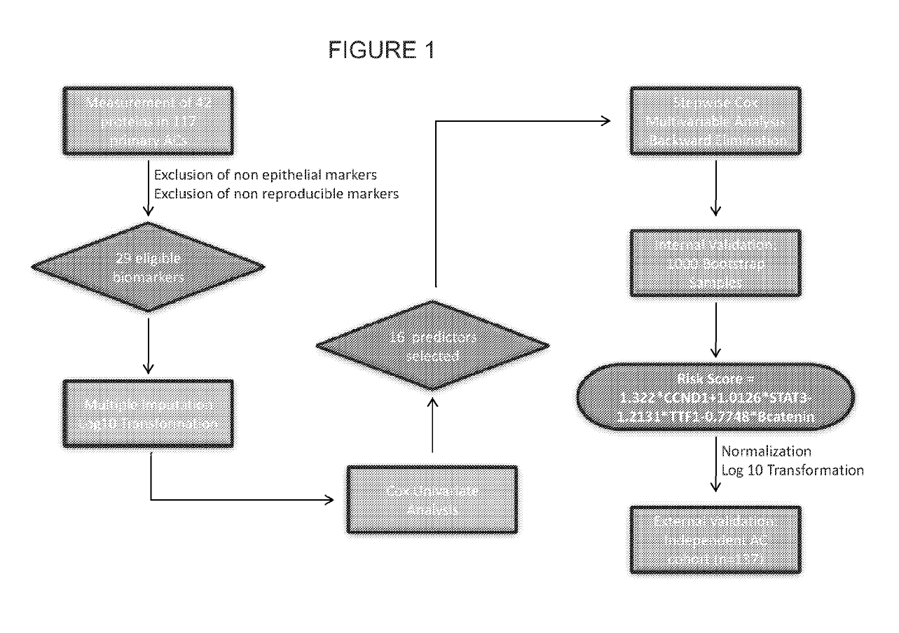

A flowchart of the statistical analysis used to develop the prognostic indicator is shown in FIG. 1. The expression of an initial set of 42 biomarkers was assessed in 117 primary lung adenocarcinoma cases in cohort 1. Biomarkers with expression in non-epithelial tissue (markers exclusively expressed on lymphocytes “contaminating” the tumor e.g. MUM1, LCK were excluded), and those for whom assay results were not reproducible (e.g. VEGF, MET, EGFR) were excluded from further analysis. There were 16 markers chosen for further analysis based on Cox Univariate analysis showing P<0.5. (Table 3).

Cox multivariate analysis of the 16 chosen markers was performed incorporating the selected biomarkers in a stepwise selection/backward elimination process. To address the issue of model over fitting, 1000 bootstrap samples were generated and a backward elimination logistic regression model was developed for each bootstrap sample; the final multivariable model included those variables that were significant at the 0.05 level (Table 3).

A risk score was generated as a linear combination of weighted expression of biomarkers in the reduced (final model) based on coefficients of the multivariate model:

1.322*CyclinD1+1.0126*STAT3−1.2131*TTF1−0.7748*Bcatenin Risk equation

The final classifier was applied to the testing and validation cohorts. All p values were based on two-sided testing and differences were considered significant at p<0.05. All statistical analyses were done using the SPSS software program (version 13.0 for Windows, SPSS Inc., Chicago, Ill.) and the R-statistics software (version 2.9.0).

### Specimen Score

Average Cyclin D1, STAT3, TTF1, and beta catenin AQUA scores from redundant cores were normalized for run-to-run variability, log 2 transformed and a score was calculated only for tumors with all 4 measurements available using the formula Risk equation:

1.322*CyclinD1+1.0126*STAT3−1.2131*TTF1−0.7748*Bcatenin. Risk equation

### Results

### Identification of Predictors

We used automated quantitative analysis to identify biomarkers that identify patients at risk for recurrence of disease. Among 42 biomarkers initially assessed, 29 were chosen for further analysis (Table 2).

TABLE 2
Eligible biomarkers used for the prognostic model development

| Biomarker | Clone/Isotype | Antigen Retrieval | Concentration | | Incubation | Positive Control | Source |
|---|---|---|---|---|---|---|---|
| Cytokeratin 5 | NCL-L-CK5/m | HIAR-pH = 6 15 min | 5.2 | μg/ml | 1 hr RT | A431 cells | Novocastra, Newcastle, UK |
| Cytokeratin 13 | DE-K13/m IgG₂ₐ, kappa | HIAR-pH = 9 15 min | 57 | μg/ml | ON 4° C. | A431 cells | Dako, Carpinteria, CA |
| Cytokeratin 14 | LL002/m IgG₃ | HIAR-pH = 6 15 min | 0.2 | μg/ml | ON 4° C. | A431 cells | Thermo Fisher, Fremont, CA |
| Cytokeratin 17 | 2D10/m IgG1 | HIAR-pH = 6 15 min | 0.1 | μg/ml | ON 4° C. | A431 cells | Abnova, Walnut, CA |
| HER2 | r polyclonal | HIAR-pH = 6 15 min | 25 | μg/ml | ON 4° C. | HER2 transfected | Dako, Carpinteria, CA |
| | | | | | | CHO cells | |
| HER3 | D11E5/r IgG | HIAR-pH = 9 15 min | 0.286 | μg/ml | ON 4° C. | HER3 transfected | Cell Signaling, Danvers, MA |
| | | | | | | BaF3 cells | |
| HER4 | SPM338/m IgG_{2b} | HIAR-pH = 6 15 min | 0.4 | μg/ml | ON 4° C. | HER4 transfected | Santa Cruz, Santa Cruz, CA |
| | | | | | | CHO cells | |
| AKT1 | 2H10/m | HIAR-pH = 6 15 min | 1/200* | | ON 4° C. | A431 cells | Cell Signaling, Danvers, MA |
| ERK | m polyclonal | HIAR-pH = 6 15 min | 1/100* | | ON 4° C. | A431 cells | Cell Signaling, Danvers, MA |
| DUSP6 | 3G2/m IgG1, kappa | HIAR-pH = 6 15 min | 0.3 | μg/ml | ON 4° C. | Pancreatic carcinoma | Novus Biologicals, Littleton, |
| | | | | | | | CO |
| STAT1 | 42H3/r IgG | HIAR-pH = 6 15 min | 1/750* | | ON 4° C. | Colon carcinoma | Cell Signaling, Danvers, MA |
| STAT2 | Y141/r IgG | HIAR-pH = 6 15 min | 1/200* | | ON 4° C. | Breast carcinoma | Novus Biologicals, Littleton, |
| | | | | | | | CO |
| STAT3 | 124H6/m | HIAR-pH = 6 15 min | 1/500* | | ON 4° C. | H1650, HCC2279 | Cell Signaling, Danvers, MA |
| | | | | | | cells | |
| mTOR | 7C10/r | HIAR-pH = 6 15 min | 1/1000* | | ON 4° C. | A431 and H1299 cells | Cell Signaling, Danvers, MA |
| pS6K | 1A5/m | HIAR-pH = 6 15 min | 1/200* | | ON 4° C. | H1299 cells | Cell Signaling, Danvers, MA |
| pS6 | 91B2/r IgG | HIAR-pH = 6 15 min | 1/400* | | ON 4° C. | Colon carcinoma, | Cell Signaling, Danvers, MA |
| | | | | | | HCC193 cells | |
| TTF1 | 8G7G3/m IgG1, kappa | HIAR-pH = 6 40 min | 20 | μg/ml | ON 4° C. | H2126 cells | Dako, Carpinteria, CA |
| E2F4 | SPM179/m IgG1, kappa | HIAR-pH = 6 15 min | 2 | μg/ml | ON 4° C. | Tonsil | Novus Biologicals, Littleton, |
| | | | | | | | CO |
| BCL2 | 124/m IgG1, kappa | HIAR-pH = 9 15 min | 2.6 | μg/ml | ON 4° C. | Lymphocytes | Dako, Carpinteria, CA |
| CC3 | 5A1/r | HIAR-pH = 6 15 min | 1/500* | | ON 4° C. | Pancreatic carcinoma | Cell Signaling, Danvers, MA |
| MENA | 21/m IgA | HIAR-pH = 6 15 min | 1 | μg/ml | ON 4° C. | A431 cells | BD Biosciences, San Jose, |
| | | | | | | | CA |
| RRM2 | 1E1/m IgG1, kappa | HIAR-pH = 6 15 min | 0.05 | μg/ml | ON 4° C. | Pancreatic carcinoma | Novus Biologicals, Littleton, |
| | | | | | | | CO |
| PTEN | 6H2.1/m IgG_{2b}, kappa | HIAR-pH = 9 15 min | 0.96 | μg/ml | ON 4° C. | H1666 cells | Dako, Carpinteria, CA |
| p53 | DO-7/m IgG_{2b}, kappa | HIAR-pH = 9 20 min | 1/5000 | | ON 4° C. | Tonsil | Dako, Carpinteria, CA |
| Ki67 | B56/m IgG1, kappa | HIAR-pH = 6 15 min | 1 | μg/ml | ON 4° C. | A549 cells | BD Biosciences, San Jose, |
| | | | | | | | CA |
| Cyclin D1 | SP4/r IgG | HIAR-pH = 6 20 min | 1/25 | | 1 hr RT | Breast carcinoma | LabVision, Fremont, CA |
| Mucin 1 | Ma695/m IgG1 | HIAR-pH = 6 20 min | 1/100 | | ON 4° C. | Breast Carcinoma | LabVision, Fremont, CA |
| Bag-1 | 2D3/m IgG₂ₐ, kappa | HIAR-pH = 6 20 min | 0.5 | μg/ml | ON 4° C. | Tonsil | Novus Biologicals, Littleton, |
| | | | | | | | CO |
| Beta-catenin | 14/m IgG1 | HIAR-pH = 6 20 min | 0.1 | μg/ml | ON 4° C. | A431 cells | BD Biosciences, San Jose, |
| | | | | | | | CA |

*The exact concentration was not provided by the manufacturer.

Of these 16 predictor biomarkers were selected as a result of Cox univariate analysis that showed P-values<0.5 (Table 3).

TABLE 3
Selected predictors in Cox univariate analysis

| | | | | 95% CI | | p |
|---|---|---|---|---|---|---|
| Biomarker | Coefficient | SE(Coef) | HR | (Lower/Upper) | | value |
| CyclinD1(tm) | 0.99 | 0.453 | 2.69 | 1.107 | 6.539 | 0.029 |
| TTF1(n) | −0.832 | 0.415 | 0.435 | 0.193 | 0.982 | 0.045 |
| pS6K(tm) | −1.586 | 0.808 | 0.205 | 0.042 | 0.998 | 0.05 |
| HER2(tm) | 0.813 | 0.538 | 2.254 | 0.785 | 6.468 | 0.131 |
| CK14(c) | 1.301 | 0.947 | 3.672 | 0.574 | 23.503 | 0.17 |
| RRM2(tm) | 0.584 | 0.44 | 1.794 | 0.757 | 4.247 | 0.184 |
| DUSP6(tm) | 1.122 | 0.849 | 3.072 | 0.581 | 16.237 | 0.186 |
| STAT3(tm) | 0.788 | 0.67 | 2.198 | 0.591 | 8.177 | 0.24 |
| CC3(tm) | 0.779 | 0.676 | 2.18 | 0.58 | 8.198 | 0.249 |
| beta Catenin(c) | −0.529 | 0.47 | 0.589 | 0.235 | 1.481 | 0.261 |
| p53(n) | 0.277 | 0.248 | 1.319 | 0.811 | 2.144 | 0.264 |
| Mucin1(tm) | 0.39 | 0.369 | 1.477 | 0.717 | 3.043 | 0.29 |
| ERK(tm) | 0.686 | 0.649 | 1.986 | 0.556 | 7.092 | 0.291 |
| BCL2(tm) | −0.46 | 0.451 | 0.631 | 0.261 | 1.529 | 0.308 |
| CK5(tm) | −0.604 | 0.696 | 0.547 | 0.14 | 2.14 | 0.386 |
| PTEN(n) | 0.424 | 0.514 | 1.527 | 0.558 | 4.181 | 0.41 |

Abbreviations: Coef; coefficient, HR; hazard ratio, CI; confidence interval, tm; tumor mask, n; nuclear, c; cytoplasmic, CC3; cleaved caspase 3

Subsequent to stepwise Cox multivariable analysis and backward elimination followed by 1000 bootstrap samples, 4 biomarkers were identified as significantly associated with risk of recurrence (Table 4).

TABLE 4
Reduced Cox multivariable model (training set).

| | | | | 95% CI | | p |
|---|---|---|---|---|---|---|
| Biomarker | Coefficient | SE(Coef) | HR | (Lower/Upper) | | value |
| Cyclin D1(tm) | 1.322 | 0.460 | 3.751 | 1.496 | 9.407 | 0.004 |
| STAT3(tm) | 1.013 | 0.640 | 2.753 | 0.777 | 9.751 | 0.110 |
| TTF1(n) | −1.213 | 0.440 | 0.297 | 0.125 | 0.706 | 0.005 |
| beta Catenin(c) | −0.775 | 0.520 | 0.461 | 0.165 | 1.285 | 0.130 |

Abbreviations: Coef; coefficient, HR; hazard ratio, CI; confidence interval, tm; tumor mask, n; nuclear, c; cytoplasmic

### Development of the Molecular Classifier (Training Cohort)

Cyclin D1, STAT3, TTF1 and beta catenin continuous AQUA scores were then incorporated in a multivariate nominal logistic regression model following a backward elimination stepwise selection process for each of the 1000 bootstrap samples; the probability of prediction of recurrence of disease was calculated as linear combination of Cyclin D1, STAT3, TTF1 and beta catenin log 2 normalized AQUA scores weighted as follows:

1.322*CyclinD1+1.0126*STAT3−1.2131*TTF1−0.7748*Bcatenin Risk equation

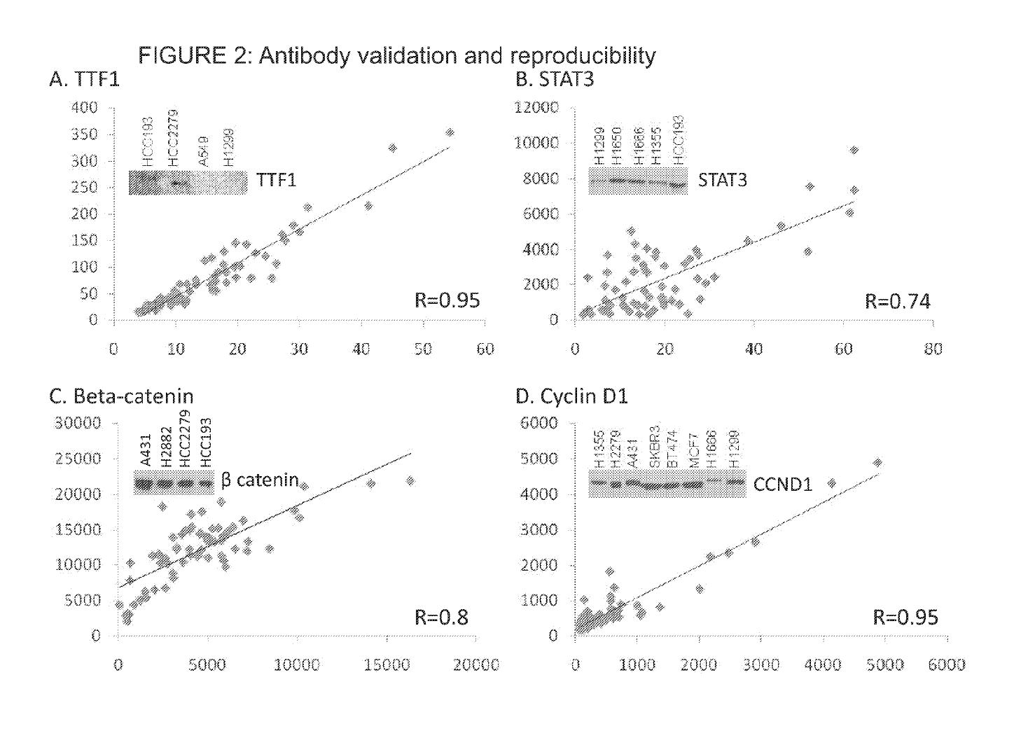

Reproducibility of assessment of biomarker assessment is shown in FIG. 2.

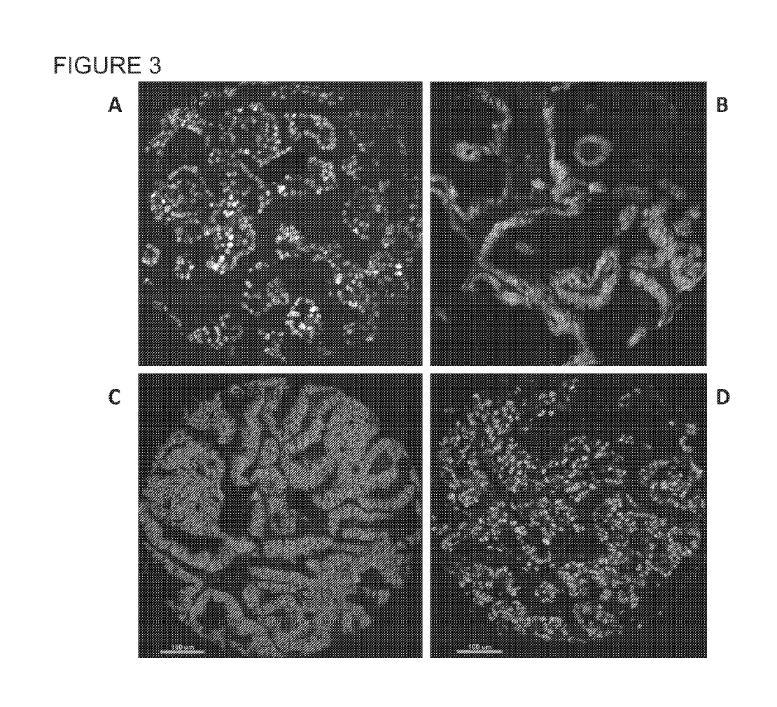

Representative staining patterns for adenocarcinoma tumors of the training cohort are shown in FIG. 3.

The performance of the classifier, as determined using Cox univariate analysis for in all patients in the training cohort, as well as for selected subsets, those that were stage 1 and stage 1A adenocarcinoma is shown in Table 5 and demonstrates the continuous risk classifier significantly predicts survival in all subpopulations.

TABLE 5
Cox univariate analyses for the classifier in all,
stage I and stage IA AC patients (training set).

| | | | | 95.0% CI for HR | | |
|---|---|---|---|---|---|---|
| | Coeffi- | | | Low- | Up- | p |
| | cient | SE(Coef) | HR | er | per | value |
| All ACs | | | | | | |
| (n = 117) | | | | | | |
| Risk Score | 1.000 | 0.251 | 2.718 | 1.661 | 4.449 | 6.96E−05 |
| (Cont.) | | | | | | |
| Stage I ACs | | | | | | |
| (n = 61) | | | | | | |
| Risk Score | 1.298 | 0.431 | 3.662 | 1.573 | 8.527 | 0.003 |
| (Cont.) | | | | | | |
| Stage IA | | | | | | |
| ACs (n = 46) | | | | | | |
| Risk Score | 1.506 | 0.499 | 4.510 | 1.696 | 11.994 | 0.003 |
| (Cont.) | | | | | | |

Abbreviations: Coef; coefficient, HR; hazard ratio, CI; confidence interval, AC; adenocarcinoma, Cont.; continuous

The performance of the continuous risk classifier as determined using Cox multivariate analysis, adjusting for clinical characteristics was assessed for all patients in the training cohort as shown in Table 6 demonstrating the continuous risk classifier significantly predicts outcome even when adjusted for stage and/or age and gender.

TABLE 6
Cox multivariate analysis for the classifier adjusting
for clinical characteristics (training set).

| | | | | | 95.0% CI for HR | | |
|---|---|---|---|---|---|---|---|
| | Vari- | Coeffi- | SE | | Low- | Up- | p |
| | able | cient | (Coef) | HR | er | per | value |
| 1^{st} | Risk Score | 0.821 | 0.278 | 2.273 | 1.319 | 3.919 | 0.003 |
| Step | (Cont.) | | | | | | |
| | Gender | 0.456 | 0.284 | 1.577 | 0.903 | 2.754 | 0.109 |
| | (M vs. F) | | | | | | |
| | Age | 0.008 | 0.014 | 1.008 | 0.981 | 1.035 | 0.576 |
| | Stage I | | | 1.000 | | | |
| | Stage II | 0.831 | 0.425 | 2.295 | 0.998 | 5.276 | 0.051 |
| | Stage III | 1.104 | 0.348 | 3.017 | 1.524 | 5.973 | 0.002 |
| | Stage IV | 0.982 | 0.435 | 2.670 | 1.138 | 6.266 | 0.024 |
| Last | Risk Score | 0.751 | 0.269 | 2.120 | 1.250 | 3.593 | 0.005 |
| Step | (Cont.) | | | | | | |
| | Stage I | | | 1.000 | | | |
| | Stage II | 0.885 | 0.406 | 2.424 | 1.094 | 5.373 | 0.029 |
| | Stage III | 1.053 | 0.326 | 2.867 | 1.512 | 5.435 | 0.001 |
| | Stage IV | 1.203 | 0.412 | 3.330 | 1.486 | 7.463 | 0.003 |

Abbreviations: Coef; coefficient, HR; hazard ratio, CI; confidence interval, Cont.; continuous, M; male, F; female

### Classifier Validation (Validation Cohorts)

The power of the molecular classifier was tested in a validation cohort of retrospectively collected cohort of 137 NSCLC patients (Yale University Lung Cancer Cohort) (Table 1). Results are shown in Table 7 indicating the continuous risk classifier developed in the training set significantly predicts survival in the validation set even when adjusted for stage and/or age and gender.

TABLE 7
Cox multivariate analysis for the classifier adjusting
for clinical characteristics (validation set).

| | | | | | 95.0% CI for HR | | |
|---|---|---|---|---|---|---|---|
| | Vari- | Coeffi- | SE | | Low- | Up- | p |
| | able | cient | (Coef) | HR | er | per | value |
| 1^{st} | Gender | −0.472 | 0.440 | 0.623 | 0.263 | 1.478 | 0.283 |
| Step | (M vs. F) | | | | | | |
| | Age | 0.008 | 0.016 | 1.008 | 0.976 | 1.041 | 0.625 |
| | Stage I | | | 1.000 | | | |
| | Stage II | 1.083 | 0.498 | 2.954 | 1.113 | 7.834 | 0.030 |
| | Stage III | 1.476 | 0.473 | 4.375 | 1.730 | 11.067 | 0.002 |
| | Stage IV | 1.933 | 0.506 | 6.908 | 2.560 | 18.640 | <0.001 |
| | Risk Score | 0.555 | 0.235 | 1.742 | 1.098 | 2.763 | 0.018 |
| | (Cont.) | | | | | | |
| Last | Stage I | | | 1.000 | | | |
| Step | Stage II | 1.077 | 0.496 | 2.934 | 1.111 | 7.754 | 0.030 |
| | Stage III | 1.430 | 0.470 | 4.180 | 1.663 | 10.504 | 0.002 |
| | Stage IV | 1.919 | 0.506 | 6.816 | 2.529 | 18.367 | <0.001 |
| | Risk Score | 0.552 | 0.245 | 1.737 | 1.074 | 2.807 | 0.024 |
| | (Cont.) | | | | | | |

Abbreviations: Coef; coefficient, HR; hazard ratio, CI; confidence interval, Cont.; continuous, M; male, F; female

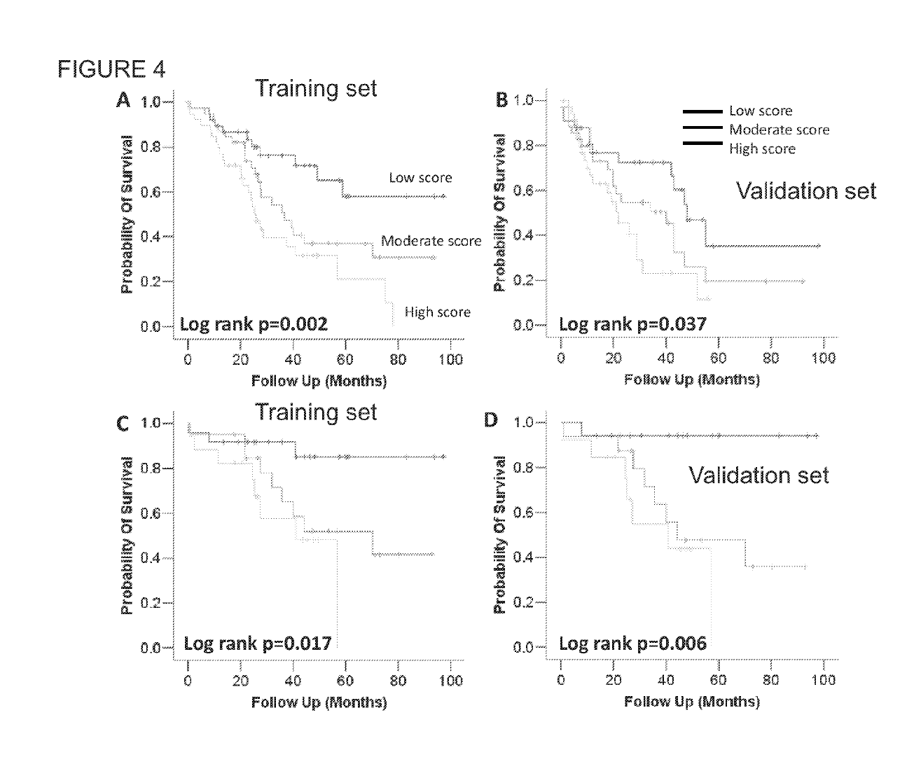

The Kaplan-Meier analysis of the prognostic algorithm in the training set is shown in FIG. 4. All adenocarcinoma patients in the training cohort could be placed into one of three prognostic groups with relatively good, moderate or poor outcome (FIG. 4A) with a statistical significance of log rank p=0.002. A subset analysis using the prognostic algorithm for adenocarcinoma patients that were stage 1 at diagnosis was also significant (FIG. 4C) log rank p=0.017. Similar results were obtained in the analysis of the validation cohort (FIGS. 4 B and D) with a log rank p=0.037 for all adenocarcinomas (FIG. 4B) and log rank p=0.006 for stage 1 patients only (FIG. 4D). Of the three prognostic groups with relatively good, moderate or poor outcome, the moderate and poor prognostic groups were combined for the results shown in FIG. 5B (log rank p=0.028). Subset analysis for Stage 1 patients is shown in FIG. 5C, and for Stage 1a patients is shown in FIG. 5D.

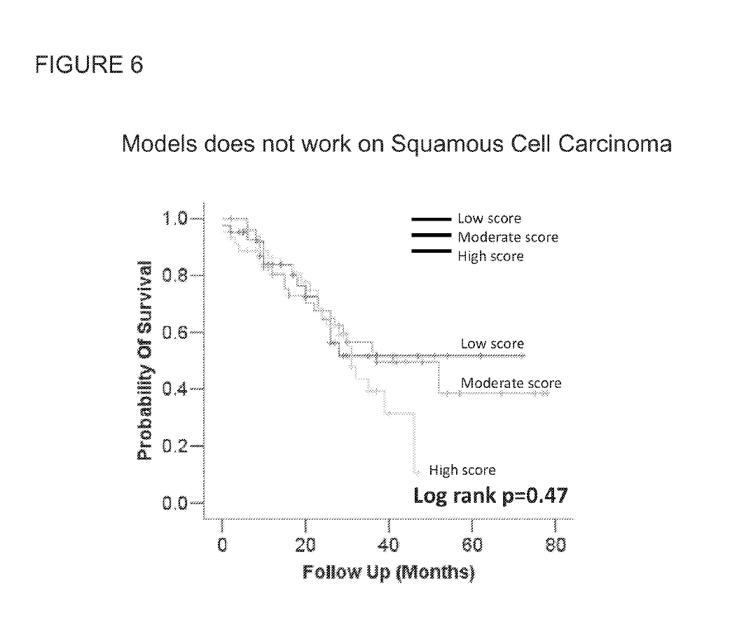

As would be expected the prognostic model is not prognostic in squamous cell lung carcinoma (FIG. 6).

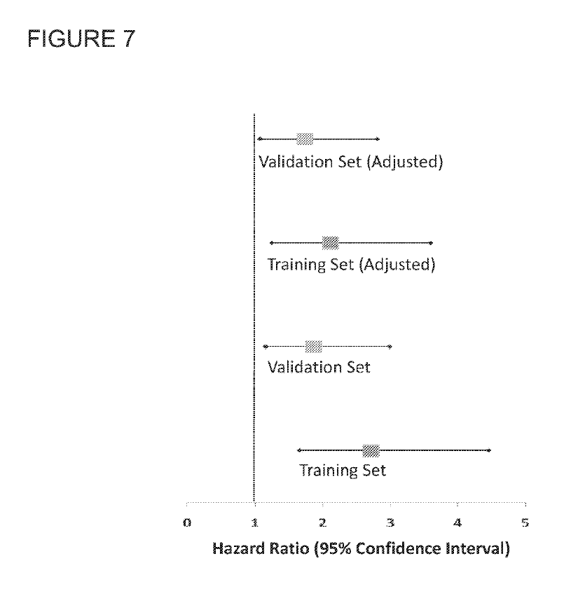

FIG. 7 represents a Forest plot summary of hazard ratio and 95% confidence interval data from Tables 5-7. All hazard ratios and 95% CIs are above one indicating that the continuous risk classifier is a significant predictor of decreased overall survival.

### Discussion

In conclusion, we have developed a highly reproducible, objective, easily applicable quantitative immuno-assay based test for prognosis of lung adenocarcinoma. This test could easily be translated into a robust diagnostic platform for broad clinical application. Moreover, we believe that identifying those patients, especially stage 1 and 1A patients at risk for disease recurrence provides the opportunity to consider adjuvant treatment and will result in the fine tuning of personalized therapy towards the goal of maximizing survival outcomes for lung cancer patients.

### PART II

### Therapeutic Treatments for NSCLC

Adjuvant platinum based chemotherapy with or without gemcitabine is a common therapy for the treatment of NSCLC, which may be guided by the use of additional assessment of biomarker expression (Reynolds J Clinical Oncology 2009, 27:5808-5815).

### Pemetrexed (Alimta, Lilly)

Mechanism of Action: Pemetrexed is chemically similar to folic acid and is in the class of chemotherapy drugs called folate antimetabolites. It works by inhibiting three enzymes used in purine and pyrimidine synthesis_thymidylate synthase (TS), dihydrofolate reductase (DHFR), and glycinamide ribonucleotide formyltransferase (McLeod, Howard L.; James Cassidy, Robert H. Powrie, David G. Priest, Mark A. Zorbas, Timothy W. Synold, Stephen Shibata, Darcy Spicer, Donald Bissett, Yazdi K. Pithavala, Mary A. Collier, Linda J. Paradiso, John D. Roberts (July-2000). “Pharmacokinetic and Pharmacodynamic Evaluation of the Glycinamide Ribonucleotide Formyltransferase Inhibitor AG2034”. Clinical Cancer Research (American. Association for Cancer Research) 6 (7): 2677-2684. PMID 10914709. http://clincancerres.aacrjournals.org/cgi/content/abstract/6/7/2677. Retrieved 2 Dec. 2008. Avendano, Carmen; Menendez, J. Carlos (16 Apr. 2008). Medicinal Chemistry of Anticancer Drugs (GARFT). By inhibiting the formation of precursor purine and pyrimidine nucleotides, pemetrexed prevents the formation of DNA and RNA, which are required for the growth and survival of both normal cells and cancer cells. Adenocarcinoma has a better response than squamous (but squamous can respond).

### Erlotinib (OSI-774, Tarceva, Genenetech & OSI in U.S.; Roche in ROW)

Mechanism of Action: erlotinib specifically targets the epidermal growth factor receptor (EGFR) tyrosine kinase, which is highly expressed and occasionally mutated in various forms of cancer. It binds in a reversible fashion to the adenosine triphosphate (ATP) binding site of the receptor (Raymond E, Faivre S, Armand J (2000). “Epidermal growth factor receptor tyrosine kinase as a target for anticancer therapy”. Drugs 60 Suppl 1: 15-23; discussion 41-2. PMID 11129168). For the signal to be transmitted, two members of the EGFR family need to come together to form a homodimer. These then use the molecule of ATP to autophosphorylate each other, which causes a conformational change in their intracellular structure, exposing a further binding site for binding proteins that cause a signal cascade to the nucleus. By inhibiting the ATP, autophosphorylation is not possible and the signal is stopped.

Response: It is reported that responses among patients with lung cancer are seen most often in females who were never smokers, particularly Asian women and those with adenocarcinoma cell type.

### Bevacuzimab (Avastin, Genentech/Roche)

Bevacizumab (trade name Avastin, Genentech/Roche) is a monoclonal antibody against vascular endothelial growth factor-A (VEGF-A) (Los M, Roodhart J M, Voest E E (April 2007). “Target practice: lessons from phase III trials with bevacizumab and vatalanib in the treatment of advanced colorectal cancer”. The Oncologist 12 (4): 443-50. doi:10.1634/theoncologist.12-4-443. PMID 17470687). It is used in the treatment of cancer, where it inhibits tumor growth by blocking the formation of new blood vessels (angiogenesis). Bevacizumab was the first clinically available angiogenesis inhibitor in the United States.

In 2006, the FDA approved bevacizumab for use in lung cancer in combination with standard first-line chemotherapy. A study conducted by the Eastern Cooperative Oncology Group (ECOG) demonstrated a 2-month improvement in overall survival in patients with Stage IIIb/IV non-small cell lung cancer (NSCLC). Due to the observance of severe pulmonary hemorrhage in patients with NSCLC with squamous histology in an earlier study, patients with such histology were excluded from the pivotal ECOG trial.

### Gemcitabine

Gemcitabine is a nucleoside analog used as chemotherapy. It is marketed as Gemzar by Eli Lilly and Company. Chemically gemcitabine is a nucleoside analog in which the hydrogen atoms on the 2′ carbons of deoxycytidine are replaced by fluorine atoms.

As with fluorouracil and other analogues of pyrimidines, the drug replaces one of the building blocks of nucleic acids, in this case cytidine, during DNA replication. The process arrests tumor growth, as new nucleosides cannot be attached to the “faulty” nucleoside, resulting in apoptosis.

1. A method for making a prognosis for a patient having a tumor associated with adenocarcimona of the lung which comprises:
a) measuring in a sample of the patient's tumor a level of expression for each of at least three biomarkers selected from the following group: thyroid transcription factor-1 (TTF1), signal transducer and activator of transcription-3 (STAT-3), beta-catenin and cyclin D1;
b) calculating a score based on the level of expression measured in step (a) for each of the biomarkers; and
c) correlating the score obtained in step (b) with a series of predetermined reference scores associated with a series of prognoses;
so as to thereby make a prognosis for the patient.

2. The method of claim 1, wherein the levels of expression of thyroid transcription factor-1 (TTF1), signal transducer and activator of transcription-3 (STAT-3), beta-catenin and cyclin D1 are determined.

3. The method of claim 1, wherein the adenocarcinoma of the lung is a stage I cancer.

4. The method of claim 1, wherein the levels of expression of thyroid transcription factor-1 (TTF1), signal transducer and activator of transcription-3 (STAT-3), beta-catenin and cyclin D1 are determined using an automated pathology system.

5. The method of claim 1, wherein the levels of expression of thyroid transcription factor-1 (TTF1), signal transducer and activator of transcription-3 (STAT-3), beta-catenin and cyclin D1 are determined using a quantitative image analysis procedure.

6. The method of claim 1, wherein the sample is a tissue sample.

7-22. (canceled)

23. A method for determining the likelihood that a patient having a tumor associated with adenocarcinoma of the lung will have a higher chance of survival after surgery from adjuvant therapy in addition to the surgery comprising:
a) measuring in a sample of the patient's tumor a level of expression for each of at least three biomarkers selected from the following group: thyroid transcription factor-1 (TTF1), signal transducer and activator of transcription-3 (STAT-3), beta-catenin and cyclin D1;
b) calculating a score based on the levels of expression determined in step (a); and
c) correlating the score obtained in step (b) with a series of predetermined reference scores associated with survival;
so as to thereby determine the likelihood that the patient will have a higher chance of survival after surgery from adjuvant therapy in addition to surgery.

24. The method of claim 23, wherein the series of predetermined reference scores are used to generate a predetermined reference score associated with chance of survival wherein there is a likelihood that the patient will have a higher chance of survival after surgery from adjuvant therapy in addition to surgery if the score obtained in step (b) is greater than the predetermined reference score.

25. The method of claim 23, wherein the levels of expression of thyroid transcription factor-1 (TTF1), signal transducer and activator of transcription-3 (STAT-3), beta-catenin and cyclin D1 are determined.

26. The method of claim 23, wherein the adenocarcinoma of the lung is a stage I cancer.

27. The method of claim 23, wherein the adjuvant therapy comprises chemotherapy.

28. The method of claim 23, wherein the levels of expression of thyroid transcription factor-1 (TTF1), signal transducer and activator of transcription-3 (STAT-3), beta-catenin and cyclin D1 are determined using an automated pathology system.

29. The method of claim 23, wherein the levels of expression of thyroid transcription factor-1 (TTF1), signal transducer and activator of transcription-3 (STAT-3), beta-catenin and cyclin D1 are determined using a quantitative image analysis procedure.

30-42. (canceled)

43. A method for determining the likelihood that a patient having a tumor associated with adenocarcinoma of the lung will have a higher chance of survival after surgery from adjuvant therapy in addition to the surgery comprising:
a) measuring a total level of expression of cyclin D1 within a nuclear compartment and non-nuclear compartments combined in cells of interest in a tumor sample from the patient;
b) measuring a total level of expression of signal transducer and activator of transcription-3 (STAT-3) within the nuclear compartment and the non-nuclear compartments combined in cells of interest in the sample from the patient;
c) measuring a level of expression of thyroid transcription factor-1 (TTF1) in the nuclear compartment in cells of interest in the sample from the patient;
d) measuring a level of expression of beta-catenin in a cytoplasmic compartment in cells of interest in the sample from the patient;
e) calculating a score based on the levels of expression determined in steps (a) through (d); and
f) correlating the score obtained in step (b) with a series of predetermined reference scores associated with survival;
so as to thereby determine the likelihood that the patient will have a higher chance of survival after surgery from adjuvant therapy in addition to surgery.

44. The method of claim 43, wherein the series of predetermined reference scores are used to generate a predetermined reference score associated with chance of survival wherein there is a likelihood that the patient will have a higher chance of survival after surgery from adjuvant therapy in addition to surgery if the score obtained in step (b) is greater than the predetermined reference score.

45. The method of claim 43, wherein the adenocarcinoma of the lung is a stage I cancer.

46. The method of claim 43, wherein the adjuvant therapy comprises chemotherapy.

47. The method of claim 43, wherein the levels of expression of thyroid transcription factor-1 (TTF1), signal transducer and activator of transcription-3 (STAT-3), beta-catenin and cyclin D1 are determined using an automated pathology system.

48. The method of claim 43, wherein the levels of expression of thyroid transcription factor-1 (TTF1), signal transducer and activator of transcription-3 (STAT-3), beta-catenin and cyclin D1 are determined using a quantitative image analysis procedure.

49. The method of claim 43, wherein the sample is a tissue sample.

50. The method of claim 43, wherein the sample is a cytology specimen.

51-73. (canceled)
